(19) United States
(12) Patent Application Publication
(10) Pub. No.: US 2026/0224693 A1
(43) Pub. Date: Aug. 6, 2026

(54) mRNA VACCINE FOR HERPES SIMPLEX VIRUS

(71) Applicant: University of Houston System, Houston, TX (US)

(72) Inventors: Shaun ZHANG, Houston, TX (US); Xinping FU, Houston, TX (US)

(21) Appl. No.: 19/152,402

(22) PCT Filed: Feb. 2, 2024
(86) PCT No.: PCT/US2024/014282

§ 371 Date: Jul. 30, 2025

Related U.S. Application Data
(60) Provisional application No. 63/482,956, filed on Feb. 2, 2023.
(60) Provisional application No. 63/490,419, filed on Mar. 15, 2023.

(51) Int. Cl. A61K 39/245; A61K 9/00; A61K 39/00; A61P 31/22; C07K 14/005; C12N 7/00

(52) U.S. Cl. CPC A61K 39/245; A61K 9/0019; A61P 31/22; C07K 14/005; C12N 7/00; A61K 2039/53; A61K 2039/54; A61K 2039/545; A61K 2039/55555; A61K 2039/70; C12N 2710/16622; C12N 2710/16634

(57) ABSTRACT

This invention relates to an mRNA-based vaccine composition for strains of herpes simplex virus (HSV), such as herpes simplex virus-2 (HSV-2). The mRNA vaccine composition comprises a combination of herpesvirus glycoprotein D (gD) and gB-pf mRNA, either alone or in combination with gE and/or gC mRNA.

This application is the U.S. national phase of International Patent Application No. PCT/US2024/014282, filed Feb. 2, 2024, which claims the benefit of U.S. Provisional Application No. 63/482,956, filed Feb. 2, 2023, and U.S. Provisional Application No. 63/490,419, filed Mar. 15, 2023, which is hereby incorporated by reference.

### SEQUENCE LISTING

The instant application contains a Sequence Listing which has been submitted electronically in XML file format and is hereby incorporated by reference in its entirety. Said XML copy, created on Mar. 13, 2024, is named 1217-0048WO.xml and is 31 kilobytes in size.

### FIELD OF THE INVENTION

This invention relates to mRNA-based vaccines for strains of herpes simplex virus (HSV), such as herpes simplex virus-2 (HSV-2).

### BACKGROUND OF THE INVENTION

Herpes simplex virus types 1 and 2 (HSV-1 and HSV-2) persist as significant health problems globally, disproportionally impacting developing countries and poor communities around the world and fueling the HIV epidemic. Vaccines are urgently needed for these infections as currently there is no effective vaccine for HSV-1, HSV-2 or HIV. HSV-1 is the primary cause of infectious blindness, while HSV-2 is the primary cause of genital ulcers globally, although HSV-1 is now more commonly identified in association with genital tract disease in developed countries. Genital herpes is a recurrent, lifelong disease that can stigmatize and psychologically impacts those affected. Infection with HSV-2 significantly increases the likelihood of acquiring and transmitting HIV, while vertical transmission of either serotype often leads to severe infant morbidity or death.

About half a billion people worldwide are living with genital herpes, and several billion have an oral herpes infection. Genital herpes is a substantial health concern worldwide, as reflected by the potential pain and discomfort suffered by people living with the infection and the associated social consequences that can profoundly affect sexual and reproductive health. Moreover, genital herpes can significantly increase the risk of HIV infection. There are nearly 600,000 new genital herpes infections each year in the United States. Nationwide, approximately 12% of persons aged 14 to 49 years have HSV-2 infection.

U.S. Pat. No. 9,284,355 describes a vaccine comprising two or more recombinant HSV proteins selected from a gD protein, a gC protein, and a gE protein, and methods of vaccinating a subject against HSV with the vaccine.

Awasthi et al., Sci. Immunol. 4, eaaw7083 (2019), 20 Sep. 2019, and International Publication No. WO 2019/035066 describe a trivalent glycoprotein vaccine containing HSV-2 entry molecule glycoprotein D (gD2) and two immune evasion molecules, glycoprotein C (gC2) and glycoprotein E (gE2). See also Egan et al., PLoS Pathog, 2020 Jul. 16(7):e1008795.

Despite the severe health burden caused by this virus infection, no vaccine is available to prevent HSV infection. The novel mRNA-based vaccine in this disclosure provides a solution to deal with this virus infection.

### SUMMARY OF THE INVENTION

HSV contains genes encoding more than a dozen glycoproteins that are assembled on the surface of the viral envelope. Four of them, gB, gD, gH, and gL are important for virus infectivity. While gD is involved in binding to the receptor for virus entry, gB contains fusion peptides that directly trigger membrane fusion between the target cell and HSV particle. gB has two structural forms—prefusion and postfusion. The status of the prefusion form of gB is important for initiating the cell-virus membrane fusion.

The inventors have tested a series of combinations of six HSV glycoproteins, namely, the regular form of glycoprotein B (gB), the prefusion fixed form of gB (gB-pf), gD, gH, gE, and gC. Mice were vaccinated with these mRNA combinations and then challenged with wild-type HSV-2 carrying the luciferase gene to evaluate the protective efficacy. A unique combination containing the prefusion form of gB was discovered to provide effective protection against HSV (such as wild-type HSV-2 infection). Specifically, it was discovered that the combination of gD and gB-pf, either alone or in combination with gE and/or gC, provides improved protection against HSV-2 compared to other combinations, such as a combination of gD, gE and gC.

The invention provides vaccine compositions comprising (a) mRNA encoding herpesvirus (such as herpes simplex virus, e.g., HSV-1 or HSV-2) glycoprotein D or an immunogenic fragment thereof (e.g., the extracellular domain region for glycoprotein D) and mRNA encoding herpesvirus glycoprotein B or an immunogenic fragment thereof (e.g., the extracellular domain region for glycoprotein B), where the mRNA for herpesvirus glycoprotein B is translated to glycoprotein B in prefusion fixed form (or at least translated into the extracellular domain region for glycoprotein B in prefusion fixed form), and (b) optionally an adjuvant. The composition may further comprise (i) mRNA encoding herpesvirus glycoprotein C or an immunogenic fragment thereof, (ii) mRNA encoding herpesvirus glycoprotein E or an immunogenic fragment thereof, or (iii) both. The invention also provides methods of vaccinating a subject (e.g., a human subject) against HSV (such as HSV-1 or HSV-2) and treating, impeding, inhibiting, reducing the incidence of, or suppressing an HSV infection or a symptom or manifestation thereof comprising administering a vaccine or immunogenic composition of the present invention. The glycoproteins (or fragments thereof) used in the vaccine compositions, immunogenic compositions and methods described herein may be HSV-1 glycoproteins (or fragments thereof) or HSV-2 glycoproteins (or fragments thereof).

One embodiment of the invention is an immunogenic composition (such as a vaccine composition) for herpes simplex virus (such as HSV-1 or HSV-2), the composition comprising (a) mRNA encoding herpesvirus glycoprotein D or an immunogenic fragment thereof and mRNA encoding herpesvirus glycoprotein B or an immunogenic fragment thereof, where the mRNA for herpesvirus glycoprotein B is translated to glycoprotein B in prefusion fixed form; and (b) optionally, an adjuvant. In one embodiment, the composition comprises mRNA encoding a mutated herpesvirus glycoprotein B or its extracellular domain region, which is stable in its pre-fusion form and does not convert to a post-fusion form upon interaction with a cellular membrane. The composition may further comprise mRNA encoding herpesvirus glycoprotein C or an immunogenic fragment thereof, mRNA encoding herpesvirus glycoprotein E or an immunogenic fragment thereof, or both. In one embodiment, the composition does not comprise mRNA encoding herpesvirus glycoprotein H or an immunogenic fragment thereof or mRNA encoding herpesvirus glycoprotein L or an immunogenic fragment thereof. In another embodiment, the composition does not comprise (i) mRNA encoding herpesvirus glycoprotein H or an immunogenic fragment thereof, (ii) herpesvirus glycoprotein H or an immunogenic fragment thereof, (iii) mRNA encoding herpesvirus glycoprotein L or an immunogenic fragment thereof, or (iv) herpesvirus glycoprotein L or an immunogenic fragment thereof. The composition may further comprise a pharmaceutically acceptable carrier. In the composition, the mRNA can be complexed with polymeric or lipid components, encapsulated in liposomes, or in lipid nanoparticles (LNPs).

The vaccine composition or immunogenic composition may be suitable for systemic, intramuscular, intradermal, subcutaneous, intravaginal, or parenteral administration or administration by intracerebroventricular or intraperitoneal injection. In one embodiment, the vaccine composition is administered to a subject at least twice.

Yet another embodiment is a method of inducing an anti-HSV immune response in a mammalian subject (e.g., a human subject) by administering to the subject an effective amount of the immunogenic composition described herein. The method may further comprise the step of administering to the subject a booster vaccination which comprises the immunogenic composition described herein.

Yet another embodiment is a method of suppressing, inhibiting, or reducing an incidence of an HSV infection (e.g., an HSV-1 infection or HSV-2 infection) in a mammalian subject (e.g., a human subject) by administering to the subject an effective amount of the immunogenic composition described herein. In one embodiment, the HSV infection is an HSV-1 infection. In one embodiment, the HSV infection is an HSV-2 infection. In one embodiment, the HSV infection is a primary HSV infection. In one embodiment, the HSV infection is a flare, recurrence, or HSV labialis or genital herpes following a primary HSV infection. In one embodiment, the HSV infection is HSV encephalitis. In one embodiment, the HSV infection is HSV keratisis or conjuctivitis. In one embodiment, the HSV infection is an HSV neonatal infection. In one embodiment, the HSV infection is an HSV eye infection. In one embodiment, the subject is HIV-infected.

Yet another embodiment is a method of inducing an anti-HSV immune response in a mammalian subject comprising providing to the subject (a) mRNA encoding herpesvirus glycoprotein D or an immunogenic fragment thereof, (b) mRNA encoding herpesvirus glycoprotein B or an immunogenic fragment thereof, and (c) optionally an adjuvant, where the mRNA for herpesvirus glycoprotein B is translated to glycoprotein B in prefusion fixed form. In one embodiment, the mRNA (a) encodes a mutated herpesvirus glycoprotein B or its extracellular domain region, which is stable in its pre-fusion form and does not convert to a post-fusion form upon interaction with a cellular membrane. In one embodiment, the method further comprises providing to the subject mRNA encoding herpesvirus glycoprotein C or an immunogenic fragment thereof, herpesvirus glycoprotein E or an immunogenic fragment thereof, or a combination thereof. In another embodiment, the subject is not administered mRNA encoding herpesvirus glycoprotein H or an immunogenic fragment thereof or mRNA encoding herpesvirus glycoprotein L or an immunogenic fragment thereof. In yet embodiment, the subject is not administered (i) mRNA encoding herpesvirus glycoprotein H or an immunogenic fragment thereof, (ii) herpesvirus glycoprotein H or an immunogenic fragment thereof, (iii) mRNA encoding herpesvirus glycoprotein L or an immunogenic fragment thereof, or (iv) herpesvirus glycoprotein L or an immunogenic fragment thereof.

Yet another embodiment is a method of vaccinating a mammalian subject against herpes simplex virus (such as HSV-1 or HSV-2) comprising providing to the mammalian subject (e.g., a human subject) mRNA encoding herpesvirus glycoprotein D or an immunogenic fragment thereof and herpesvirus glycoprotein B or an immunogenic fragment thereof, where the mRNA for herpesvirus glycoprotein B is translated to glycoprotein B in prefusion fixed form. In one embodiment, the mRNA herpesvirus glycoprotein B or an immunogenic fragment thereof is a mRNA which encodes a mutated herpesvirus glycoprotein B or its extracellular domain region, which is stable in its pre-fusion form and does not convert to a post-fusion form upon interaction with a cellular membrane. In one embodiment, the method further comprises providing mRNA encoding herpesvirus glycoprotein C or an immunogenic fragment thereof, herpesvirus glycoprotein E or an immunogenic fragment thereof, or a combination thereof. In one embodiment, the subject is not administered mRNA encoding herpesvirus glycoprotein H or an immunogenic fragment thereof or herpesvirus glycoprotein L or an immunogenic fragment thereof.

The mRNA may be provided to the subject by administration of an immunogenic or vaccine composition comprising the mRNA, for example, as described herein. In one embodiment, the composition does not comprise mRNA encoding herpesvirus glycoprotein H or an immunogenic fragment thereof or herpesvirus glycoprotein L or an immunogenic fragment thereof. In another embodiment, the composition does not comprise (i) mRNA encoding herpesvirus glycoprotein H or an immunogenic fragment thereof, (ii) herpesvirus glycoprotein H or an immunogenic fragment thereof, (iii) mRNA encoding herpesvirus glycoprotein L or an immunogenic fragment thereof, or (iv) herpesvirus glycoprotein L or an immunogenic fragment thereof. In one embodiment, the composition is administered to the subject at least twice.

The mRNA may be complexed with polymeric or lipid components, encapsulated in liposomes, or in lipid nanoparticles (LNPs).

In any of the methods described herein, the mRNA may be administered systemically, intramuscularly, intradermally, subcutaneously, intravaginally, or parenterally or by intracerebroventricular or intraperitoneal injection.

In any of the compositions and methods described herein, the actual glycoprotein can be substituted in the composition or method step for the mRNA encoding the same glycoprotein. For instance, one embodiment is an immunogenic composition comprising (a) a mutated herpesvirus glycoprotein B or an immunogenic fragment thereof (such as its extracellular domain region), which is stable in its pre-fusion form and does not convert to a post-fusion form upon interaction with a cellular membrane and (b) herpesvirus glycoprotein D or an immunogenic fragment thereof. Another embodiment is an immunogenic composition comprising (a) a mutated herpesvirus glycoprotein B or an immunogenic fragment thereof (such as its extracellular domain region), which is stable in its pre-fusion form and does not convert to a post-fusion form upon interaction with a cellular membrane and (b) mRNA encoding herpesvirus glycoprotein D or an immunogenic fragment thereof.

Yet another embodiment is a mutated herpesvirus glycoprotein B or an immunogenic fragment thereof (such as its extracellular domain region), which is stable in its pre-fusion form and does not convert to a post-fusion form upon interaction with a cellular membrane. The glycoprotein can be an HSV-2 glycoprotein B, such as the HSV-2 glycoprotein B sequence set forth in SEQ ID NO:1 with a mutation at amino acid position 513 (such as H513P). In another embodiment, the glycoprotein can be an HSV-1 glycoprotein B, such as the HSV-1 glycoprotein B sequence set forth in SEQ ID NO:5 with a mutation at amino acid position 516 (such as H516P).

Yet another embodiment is an immunogenic composition comprising an mRNA encoding a mutated herpesvirus glycoprotein B or immunogenic fragment described herein (for example, as described in the paragraph above). The immunogenic composition may further comprises an adjuvant. The immunogenic composition may further comprises an mRNA encoding herpesvirus glycoprotein D or an immunogenic fragment thereof and optionally, (i) mRNA encoding herpesvirus glycoprotein C or an immunogenic fragment thereof, (ii) mRNA encoding herpesvirus glycoprotein E or an immunogenic fragment thereof, or (iii) both.

Yet another embodiment is a nucleic acid (such as an RNA or mRNA) encoding a mutated herpesvirus glycoprotein B described herein.

### BRIEF DESCRIPTION OF THE DRAWINGS

For a more complete understanding of the present invention, including features and advantages, reference is now made to the detailed description of the invention along with the accompanying figures:

FIG. 1A is a diagram of HSV-2 showing surface glycoproteins.

FIG. 1B is a diagram of the HSV-2 surface in relation to a cell membrane and the four essential viral glycoproteins (gD, gB, gH and gL) and their role in initiating HSV infection.

FIG. 1C is a diagram of the HSV-2 gB glycoprotein in its prefusion configuration and postfusion configuration.

FIG. 2 is a diagram showing the action mechanism of HSV-2 gC at antagonizing the complement system by binding and covering the C3b, and gE at antagonizing the anti-HSV antibodies by binding to and covering the Fc region of the antibodies.

FIG. 3A is a graph showing qPCR of day 1 (D1) vaginal swabs, measuring DNA copies of HSV-2, after mice vaccinated with the indicated combination of mRNAs were challenged intravaginally with HSV-2 containing the luciferase gene.

FIG. 3B is a graph showing qPCR results of day 1 (D1) vaginal swabs, logarithmically measuring DNA copies of HSV-2.

FIG. 4A is a graph showing qPCR results of day 3 (D3) vaginal swabs, measuring DNA copies of HSV-2.

FIG. 4B is a graph showing qPCR results of day 3 (D3) vaginal swabs, logarithmically measuring DNA copies of HSV-2.

FIG. 5A is a graph showing qPCR results of day 5 (D5) vaginal swabs, measuring DNA copies of HSV-2.

FIG. 5B is a graph showing qPCR results of day 5 (D5) vaginal swabs, logarithmically measuring DNA copies of HSV-2.

FIG. 6A is a graph showing qPCR results of day 7 (D7) vaginal swabs, measuring DNA copies of HSV-2.

FIG. 6B is a graph showing qPCR results of day 7 (D7) vaginal swabs, logarithmically measuring DNA copies of HSV-2.

FIG. 7 shows IVIS in vivo images of mice inoculated with mRNA vaccines of the present technology at day 2 (D2), day 4 (D4), and (D6), showing luciferase expression from the challenged HSV-2 infection in tissue.

FIG. 8 shows the neutralization effect of the sera collected from vaccinated mice on the infectivity of wild type HSV-2. The number of plaques inversely correlates with the neutralization efficiency of the immune sera, i.e.s the smaller the plaque number, the more effective the sera were at neutralizing the virus infection.

### DETAILED DESCRIPTION OF THE INVENTION

### Introduction

### Definitions

The term “Herpes Simplex Virus” or “HSV” as used herein refers to an enveloped, icosahedral, double-stranded DNA virus that infects mammals, including humans. Wild-type HSV infects and replicates in both terminally differentiated non-dividing cells and dividing cells.

“HSV-1” refers, in another embodiment, to a Herpes Simplex Virus-1. In another embodiment, the term refers to a KOS strain. In another embodiment, the term refers to an F strain. In another embodiment, the term refers to an NS strain. In another embodiment, the term refers to a CL101 strain. In another embodiment, the term refers to a “17” strain. In another embodiment, the term refers to a “17+syn” strain. In another embodiment, the term refers to a MacIntyre strain. In another embodiment, the term refers to an MP strain. In another embodiment, the term refers to an HF strain. In another embodiment, the term refers to any other HSV-1 strain known in the art.

“HSV-2” refers, in another embodiment, to a Herpes Simplex Virus-2. In another embodiment, the term refers to an HSV-2 333 strain. In another embodiment, the term refers to a 2.12 strain. In another embodiment, the term refers to an HG52 strain. In another embodiment, the term refers to an MS strain. In another embodiment, the term refers to a G strain. In another embodiment, the term refers to an 186 strain. In another embodiment, the term refers to any other HSV-2 strain known in the art.

The term “encoding” refers, in one embodiment, to an RNA molecule that contains a gene that encodes the protein of interest. In another embodiment, the RNA molecule comprises a protein coding sequence that encodes the protein of interest. In another embodiment, one or more other proteins is also encoded. In another embodiment, the protein of interest is the only protein encoded. Each possibility represents a separate embodiment of the present invention.

The term “immunogenic fragment” refers, in one embodiment, to a portion of a protein that is immunogenic and elicits a protective immune response when administered to a subject. In one embodiment, the immunogenic fragment of a glycoprotein is the extracellular domain region of the glycoprotein.

In one embodiment, the term “immunogenicity” or “immunogenic” is used herein to refer to the innate ability of a protein, peptide, nucleic acid, antigen or organism to elicit an immune response in an animal when the protein, peptide, nucleic acid, antigen or organism is administered to the animal.

In one embodiment, the term “functional” within the meaning of the invention, is used herein to refer to the innate ability of a protein, peptide, nucleic acid, fragment or a variant thereof to exhibit a biological activity or function. In one embodiment, such a biological function is its binding property to an interaction partner, e.g., a membrane-associated receptor, and in another embodiment, its trimerization property. In the case of functional fragments and the functional variants of the invention, these biological functions may in fact be changed, e.g., with respect to their specificity or selectivity, but with retention of the basic biological function.

In one embodiment, the term “fragment” is used herein to refer to a protein or polypeptide that is shorter or comprises fewer amino acids than the full length protein or polypeptide. In another embodiment, the term “fragment” refers to a nucleic acid encoding the protein fragment that is shorter or comprises fewer nucleotides than the full length nucleic acid. In another embodiment, the fragment is an N-terminal fragment. In another embodiment, the fragment is a C-terminal fragment. In one embodiment, the fragment is an intrasequential section of the protein, peptide, or nucleic acid. In another embodiment, the fragment is an immunogenic intrasequential section of the protein, peptide or nucleic acid. In another embodiment, the fragment is a functional intrasequential section within the protein, peptide or nucleic acid. In another embodiment, the fragment is an N-terminal immunogenic fragment. In one embodiment, the fragment is a C-terminal immunogenic fragment. In another embodiment, the fragment is an N-terminal functional fragment. In another embodiment, the fragment is a C-terminal functional fragment. In another embodiment, the fragment contains pieces of the protein linked together or pieces of multiple proteins linked together.

In one embodiment, an “immunogenic fragment” of a protein refers to a portion of the protein that is immunogenic, in one embodiment and in another embodiment, elicits a protective immune response when administered to a subject.

The term “composition” refers to at least one mRNA and, optionally, further excipients. The term “composition” thus comprises any mixture of mRNAs encoding the glycoproteins described herein, irrespective of whether the mRNAs are mono-, bi- or multicistronic. The term “composition” preferably relates to the at least one mRNA together with at least one other suitable substance. In general, the composition may be a pharmaceutical composition, which is designed for use in the medical field. Accordingly, the composition typically comprises at least one further excipient, which is pharmaceutically acceptable and which may be selected, for example, from carriers, vehicles and the like. The “composition” may be a liquid or a dry composition. If the composition is liquid, it will be preferably an aqueous solution or dispersion of the at least one mRNA. If the “composition” is a dry composition, it will typically be a lyophilized composition of at least one mRNA. The term “composition”, as used herein, further refers to the at least one mRNA of the invention in combination with a further active ingredient. Preferably, the composition is an immunostimulatory composition, i.e. a composition comprising at least one component, which is able to induce an immune response or from which a component, which is able to induce an immune response, is derivable. In this context, the immune response may be the result of the adaptive and/or of the innate immune system.

The term “effective” or “therapeutically effective” as used herein refers to suppressing or inhibiting an exacerbation in symptoms, inhibiting, suppressing, or preventing onset of a disease, inhibiting, suppressing, or preventing spread of disease, amelioration of at least one symptom of disease, or a combination thereof.

The term “nucleic acid” is used in its broadest sense and encompasses any compound and/or substance that includes a polymer of nucleotides, or derivatives or analogs thereof.

The term “patient” or “subject” refers to a mammal such as human or a domestic animal (e.g., a dog or cat). In a preferred embodiment, the patient or subject is a human.

The term “sera” or “serum” refers to the fluid from blood that remains when hematocytes and clotting proteins are removed.

The phrases “pharmaceutically” or “pharmacologically acceptable” as used herein refer to molecular entities and compositions that do not produce an adverse, allergic or other untoward reaction when administered to an animal, or human, as appropriate. The phrase “pharmaceutically acceptable carrier” includes any and all solvents, dispersion media, coatings, antibacterial and antifungal agents, isotonic and absorption delaying agents and the like.

The term “unit dose” refers to a physically discrete unit suitable for use in a subject, each unit containing a predetermined quantity of the therapeutic composition calculated to produce the desired response in association with its administration, i.e., the appropriate route and treatment regimen.

In one embodiment, a “flare” or “recurrence” refers to reinfection of skin tissue following latent neuronal HSV infection. In another embodiment, the terms refer to reactivation of HSV after a latency period. In another embodiment, the terms refer to symptomatic HSV lesions following a non-symptomatic latency period.

In one embodiment, any reference to HSV in the composition and methods of the instant invention refers, in one embodiment, to HSV-1, and in another embodiment, to HSV-2, and in another embodiment, to both HSV-1 and HSV-2, and in another embodiment, to HSV-1 or HSV-2.

### Immunogenic Compositions

The invention provides an immunogenic composition comprising (a) mRNA encoding herpesvirus (such as herpes simplex virus, e.g., HSV-1 or HSV-2) glycoprotein D or an immunogenic fragment thereof and mRNA encoding herpesvirus glycoprotein B or an immunogenic fragment thereof, where the mRNA for herpesvirus glycoprotein B is translated to glycoprotein B in prefusion fixed form, and (b) optionally, an adjuvant.

The composition may further comprise (i) mRNA encoding herpesvirus glycoprotein C or an immunogenic fragment thereof, (ii) mRNA encoding herpesvirus glycoprotein E or an immunogenic fragment thereof, or (iii) both.

In one embodiment, the composition comprises (a) (i) mRNA encoding herpesvirus (such as herpes simplex virus, e.g., HSV-1 or HSV-2) glycoprotein D or an immunogenic fragment thereof, (ii) mRNA encoding herpesvirus glycoprotein B or an immunogenic fragment thereof, where the mRNA for herpesvirus glycoprotein B is translated to glycoprotein B in prefusion fixed form, and (iii) mRNA encoding herpesvirus glycoprotein C or an immunogenic fragment thereof, and (b) optionally, an adjuvant.

In another embodiment, the composition comprises (a) (i) mRNA encoding herpesvirus (such as herpes simplex virus, e.g., HSV-1 or HSV-2) glycoprotein D or an immunogenic fragment thereof, (ii) mRNA encoding herpesvirus glycoprotein B or an immunogenic fragment thereof, where the mRNA for herpesvirus glycoprotein B is translated to glycoprotein B in prefusion fixed form, and (iii) mRNA encoding herpesvirus glycoprotein E or an immunogenic fragment thereof, and (b) optionally, an adjuvant.

In yet another embodiment, the composition comprises (a) (i) mRNA encoding herpesvirus (such as herpes simplex virus, e.g., HSV-1 or HSV-2) glycoprotein D or an immunogenic fragment thereof, (ii) mRNA encoding herpesvirus glycoprotein B or an immunogenic fragment thereof, where the mRNA for herpesvirus glycoprotein B is translated to glycoprotein B in prefusion fixed form, (iii) mRNA encoding herpesvirus glycoprotein C or an immunogenic fragment thereof, and (iv) mRNA encoding herpesvirus glycoprotein E or an immunogenic fragment thereof, and (b) optionally, an adjuvant.

In one embodiment, the composition does not comprise mRNA encoding herpesvirus glycoprotein H or an immunogenic fragment thereof or mRNA encoding herpesvirus glycoprotein L or an immunogenic fragment thereof. In another embodiment, the composition does not comprise (i) mRNA encoding herpesvirus glycoprotein H or an immunogenic fragment thereof, (ii) herpesvirus glycoprotein H or an immunogenic fragment thereof, (iii) mRNA encoding herpesvirus glycoprotein L or an immunogenic fragment thereof, or (iv) herpesvirus glycoprotein L or an immunogenic fragment thereof.

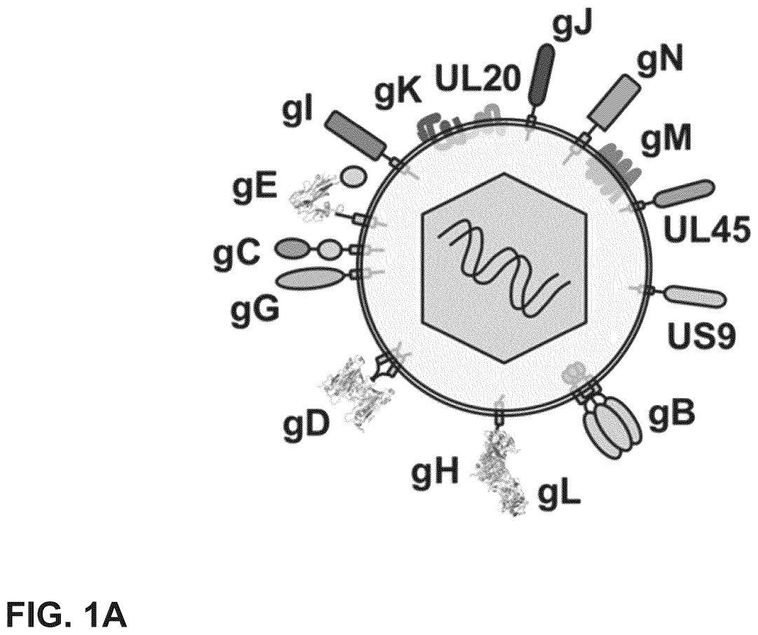

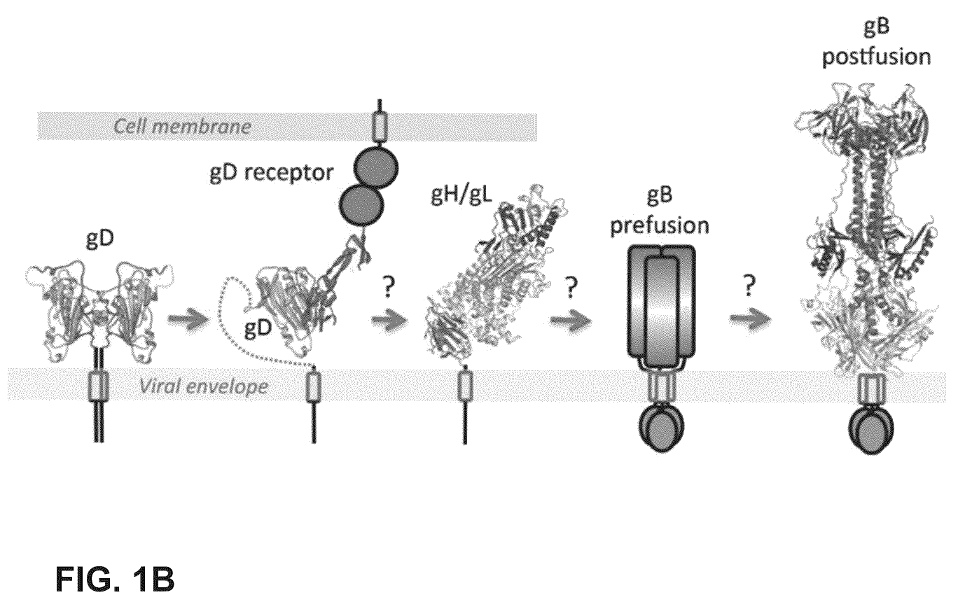

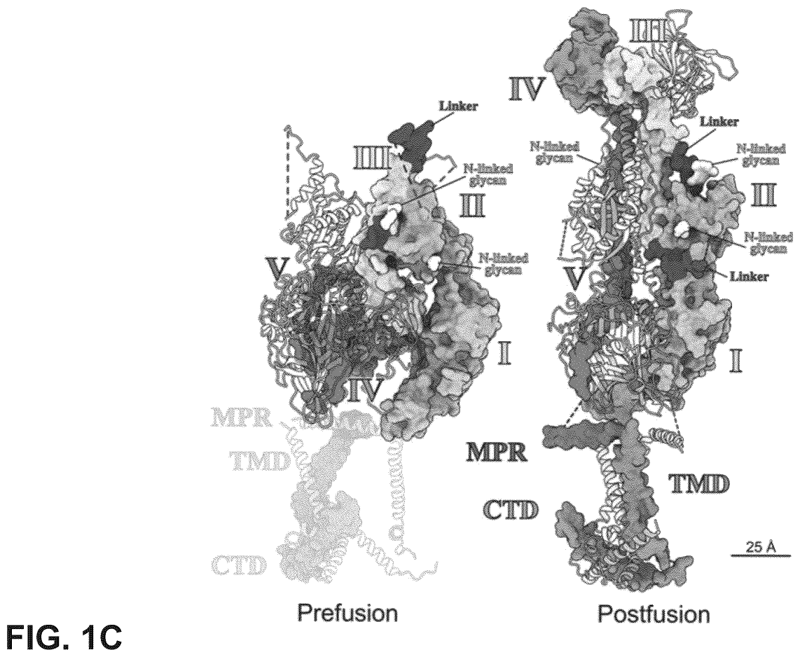

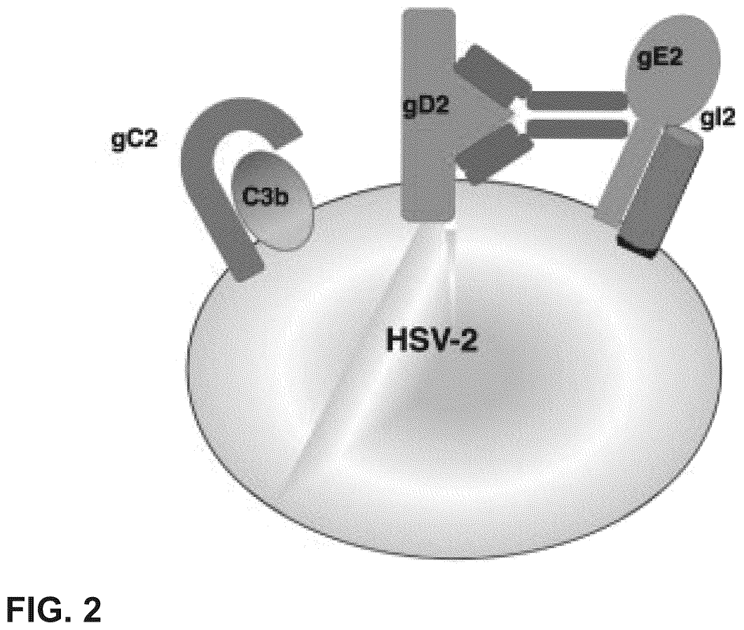

As described herein, HSV contains genes encoding more than a dozen glycoproteins that are assembled on the surface of the viral envelope. FIG. TA is a diagram of HSV-2, showing surface glycoproteins. Of the glycoproteins that are shown, four of them, gB, gD, gH, and gL are essential for virus infectivity. FIG. 1B is a diagram of the HSV-2 glycoproteins, gB, gD, gH, and gL, on the viral envelope in relation to a cell membrane. As shown, while gD is involved in binding to a cell receptor for virus entry, gB contains fusion peptides that directly trigger membrane fusion between the target cell and HSV particle. The binding of gD with the receptor initiates the gD to interact with the gH/gL complex. This triggers the gH/gL to interact with gB, causing the structural change of gB (from pre-fusion form to post-fusion form), resulting in the exposure of the fusion peptide and the subsequent membrane fusion between viral and cellular membranes. FIG. 2 is a diagram showing how gC and gE enable the virus to escape the complement-mediated innate antiviral mechanism and antiviral IgG mediated antibody-mediated phagocytosis, respectively. Hence, even though neither gC nor gE is directly involved in the HSV-2 infection process, antibodies blocking either or both of these two glycoproteins will also help to protect the body against HSV infection, an objective in the development of the vaccines disclosed herein.

mRNA for Glycoproteins

Glycoprotein (gB) has two structural forms: prefusion and postfusion. FIG. 1C is a diagram of the HSV-2 gB glycoprotein in its prefusion configuration and postfusion configuration. The prefusion form of gB (gB-pf) facilitates initiation of the cell-virus membrane fusion. The prefusion and postfusion forms of gB are structurally quite different. Prior to the present invention, the design in the art was to use the postfusion form of gB as a vaccine to induce antibodies, which may not bind to the prefusion form of gB and limit their efficacy. The amino acid sequence for HSV-2 glycoprotein gB-pf is provided in SEQ ID NO:1 (GenBank No. AAA66440.1).

In one embodiment, the composition includes an mRNA encoding HSV-2 gB-pf (SEQ ID NO:1). A corresponding DNA sequence for in vitro preparation of this mRNA is shown in SEQ ID NO:2. In another embodiment, the composition includes the mRNA which encodes the extracellular domain region of HSV-2 gB-pf (corresponding to amino acids 1-792 of SEQ ID NO:1). In one embodiment, the composition includes the mRNA counterpart to the DNA sequence for HSV-2 gB-pf provided in SEQ ID NO:2 or the portion of the DNA which encodes the extracellular domain region of HSV-2 gB-pf (corresponding to amino acids 1-792 of SEQ ID NO:1).

In another embodiment, the immunogenic composition comprises an mRNA encoding HSV-2 glycoprotein gB which is fixed in its prefusion form. In another embodiment, the composition includes an mRNA which encodes a mutated HSV-2 gB-pf in which the mutation fixes gB so that it remains in its pre-fusion form (gB-pf). For instance, the encoded HSV-2 gB-pf may have a mutation at amino acid position 513, for example, H513P as shown in SEQ ID NO:3, in order to fix it in pre-fusion form. This mutation makes the encoded gB protein fixed in the prefusion form whenever it is produced in the body from translation of the delivered mRNA. A corresponding DNA sequence for in vitro preparation of this mRNA is shown in SEQ ID NO:4. In yet another embodiment, the composition includes mRNA which encodes the extracellular domain region of the mutated HSV-2 gB-pf, such as an mRNA which encodes amino acids 1-792 of SEQ ID NO:3. In one embodiment, the composition includes the mRNA counterpart to the DNA sequence provided in SEQ ID NO:4 for the mutated HSV-2 gB-pf or the portion of the DNA which encodes the extracellular domain region of HSV-2 gB-pf (corresponding to amino acids 1-792 of SEQ ID NO:3).

In another embodiment, the composition includes an mRNA encoding HSV-1 gB-pf (SEQ ID NO:5) (UniProtKB Accession No. P10211.1) or the extracellular domain region of HSV-1 gB-pf (corresponding to amino acids 1-696 of SEQ ID NO:5). In another embodiment, the composition comprises an mRNA encoding HSV-1 glycoprotein gB which is fixed in its prefusion form. In another embodiment, the composition includes an mRNA which encodes a mutated HSV-1 gB-pf in which the mutation fixes gB so that it remains in its pre-fusion form (gB-pf). For instance, the encoded HSV-1 gB-pf may have a mutation at amino acid position 516, for example, H516P as shown in SEQ ID NO:6, in order to fix it in pre-fusion form.

In one embodiment, the composition includes an mRNA encoding HSV-2 gD (SEQ ID NO:7) or an extracellular domain region thereof (corresponding to amino acids 1-365 of SEQ ID NO:7). A corresponding DNA sequence for in vitro preparation of this mRNA is shown in SEQ ID NO:8. In another embodiment, the composition comprises an mRNA encoding a fragment of HSV-2 glycoprotein gD2 (amino acids 26-331) set forth in SEQ ID NO:9.

In yet another embodiment, the composition includes an mRNA encoding HSV-1 gD (gD1) or an extracellular domain region thereof. In one embodiment, the HSV-1 gD encoded by the mRNA utilized in the methods and compositions described herein comprises the amino acid sequences as set forth in any one of the following GenBank Accession Numbers: AAL90884.1 (KHS2 strain), AAL90883.1 (KHS1 strain), AAK93950.1 (F strain), AAB59754.1 (F strain), AAA19631.1, AAA19630.1, AAA19629.1, A1Z0Q5.2, AAA45780.1 AAA45785.1, AAA45786.1, AAA96682.1, AAK19597.1, AAN74642.1, ABI63524.1 ABM52978.1, ABM52979.1, ABM52980.1, ABM52981.1, ABM66847.1, ABM66848.1 ACM62295.1, ADD60053.1, ADD60130.1, ADM22389.1, ADM22466.1, ADM22542.1 ADM22619.1, ADM22696.1, ADM22773.1, ADM22849.1, ADM22926.1, ADM23003.1, ADM23079.1, ADM23155.1, ADM23231.1, ADM23309.1, ADM23383.1, ADM23457.1, ADM23531.1, ADM23605.1, ADM23680.1, ADM23755.1, ADM23831.1, AEQ77097.1 AER37647.1, AER37715.1, AER37786.1, AER37857.1, AER37929.1, AER38000.1, AER38070.1 AFE62894.1, AFH41180.1, AFI23657.1, AFK50415.1 AFP86430.1 AGZ01928.1 AIR95858.1, AJE60009.1, AJE60080.1, AJE60151.1, AJE60222.1, AJE60293.1 AJE60439.1, AKE48645.1, AKG59246.1, AKG59318.1, AKG59391.1, AKG59462.1, AKG59536.1, AKG59609.1, AKG59682.1, AKG59755.1, AKG59826.1, AKG59898.1, AKG59972.1, AKG60046.1, AKG60118.1, AKG60189.1, AKG60261.1, AKG60334.1, AKG60404.1 AKG60474.1, AKG60546.1, AKG60620.1, AKG60692.1, AKG60763.1, AKG60835.1, AKG60906.1, AKG60978.1, AKG61050.1, AKG61123.1, AKG61194.1, AKG61267.1, AKG61339.1, AKG61411.1, AKG61484.1, AKG61556.1, AKG61629.1, AKG61703.1, AKG61774.1, AKG61847.1, AKG61920.1, AKG61993.1, AKH80463.1, AKH80536.1, ALM22635.1, ALM22709.1, ALM22783.1, ALM22857.1, ALO18662.1, ALO18738.1, AMB65662.1, AMB65735.1, AMB65809.1, AMB65885.1, AMB65956.1, AMN09832.1, ANN83964.1, ANN84041.1, ANN84117.1, ANN84194.1, ANN84271.1, ANN84348.1, ANN84424.1, ANN84500.1, ANN84577.1, ANN84653.1, ANN84730.1, ANN84806.1, ANN84883.1, ANN84959.1, ANN85036.1, ANN85112.1, ANN85187.1, ANN85264.1, ANN85341.1, ANN85416.1, ANN85494.1, ANN85571.1, ANN85648.1, ANN85724.1, ANN85801.1, AOY34093.1, AOY34141.1, AOY34243.1, AOY34271.1, AOY34337.1, AOY36685.1, ARB08957.1, AR037961.1, AR037962.1, AR037963.1, AR037964.1, AR037965.1, AR037966.1, AR037967.1, AR037968.1, AR037969.1, ARO37970.1, AR037971.1, AR037972.1, AR037973. L AR037974.1, AR037975.1, AR037976.1, AR037977.1, AR037978.1, AR037979.1, ARO37980.1 AR037981.1, AR037982.1, AR037983.1, AR037984.1, AR037985.1, AR037986.1, AR037987.1, AR037988.1, AR037989.1, ARO37990.1, AR037991.1, AR037992.1, AR037993.1, AR037994.1, AR037995.1, AR037996.1, AR037997.1, AR037998.1, AR037999.1 ASM47664.1 ASM47741.1 ASM47818.1, ASM47893.1, BAM73419.1, CAA26060.1, CAA32283.1, CAA32284.1, CAA32289.1, CAA38245.1, CAT05431.1, P06476.1, P36318.1, P57083.1, P68331.1, Q05059.1, Q69091.1, SBO07792.1, SB007819.1, SBO07855.1 SB007869.1, SB007887.1, SB007908.1, SBS69553.1, SBS69561.1, SBS69579.1, SBS69625.1, SBS69688.1, SBS69694.1, SBS69717.1, SBS69727.1, SBS69811.1, SBT69395.1, SCL76902.1, VGBEDZ, or YP_009137141.1.

In one embodiment, the immunogenic composition comprises an mRNA encoding HSV-2 gE. In another embodiment, the composition comprises an mRNA encoding the HSV-2 gE set forth in SEQ ID NO:10. A corresponding DNA sequence for in vitro preparation of this mRNA is shown in SEQ ID NO:11. In another embodiment, the composition comprises an mRNA encoding a fragment of HSV-2 glycoprotein gE2 (amino acids 24-405) set forth in SEQ ID NO:12. In yet another embodiment, the immunogenic composition comprises an immunogenic fragment of HSV-2 gE, such as the extracellular domain region of HSV-2 gE (corresponding to amino acids 1-435 of SEQ ID NO:10).

In one embodiment, the composition includes an mRNA encoding HSV-1 gE or an extracellular domain region thereof. In one embodiment, the HSV-1 gE encoded by the mRNA utilized in the methods and compositions described herein comprises the amino acid sequences as set forth in any one of the following GenBank Accession Numbers: AAA45779.1, AAA96680.1 ABI63526.1, ACM62297.1, ADD60055.1, ADD60132.1, ADM22391.1, ADM22468.1, ADM22544.1, ADM22621.1, ADM22698.1, ADM22775.1, ADM22851.1, ADM22928.1, ADM23005.1, ADM23081.1, ADM23157.1, ADM23233.1, ADM23311.1, ADM23385.1, ADM23459.1, ADM23533.1, ADM23607.1, ADM23682.1, ADM23757.1, ADM23833.1, ADN34689.1, ADN34692.1, ADN34695.1, AEQ77099.1, AER37649.1, AER37717.1, AER37788.1, AER37859.1, AER37931.1, AER38002.1, AER38072.1, AFA36179.1, AFA36180.1, AFA36181.1, AFA36182.1, AFA36183.1, AFA36184.1, AFA36185.1, AFA36186.1, AFA36187.1, AFA36188.1, AFA36189.1, AFA36190.1, AFA36191.1, AFA36192.1, AFA36193.1, AFA36194.1, AFA36195.1, AFA36196.1, AFA36197.1, AFA36198.1, AFA36199.1, AFA36200.1, AFA36201.1, AFA36202.1, AFA36203.1 AFE62896.1, AFI23659.1, AFK50417.1, AFP86432.1, AGZ01930.1, AIR95859.1, AJE60011.1, AJE60082.1, AJE60153.1, AJE60224.1, AJE60295.1, AKE48647.1, AKE98373.1, AKE98374.1, AKE98375.1, AKE98376.1, AKE98377.1, AKE98378.1, AKE98379.1, AKE98380.1, AKE98381.1, AKE98382.1, AKE98383.1, AKE98384.1, AKE98385.1, AKE98386.1, AKE98387.1, AKE98388.1, AKE98389.1, AKE98390.1, AKE98391.1, AKE98392.1, AKE98393.1, AKG59248.1, AKG59320.1, AKG59393.1, AKG59464.1, AKG59538.1, AKG59611.1, AKG59684.1, AKG59757.1, AKG59828.1, AKG59900.1, AKG59974.1, AKG60048.1, AKG60120.1, AKG60191.1, AKG60263.1, AKG60336.1, AKG60406.1, AKG60476.1, AKG60548.1, AKG60622.1, AKG60694.1, AKG60765.1, AKG60837.1, AKG60908.1, AKG60980.1, AKG61052.1, AKG61125.1, AKG61196.1, AKG61269.1, AKG61341.1, AKG61413.1, AKG61486.1, AKG61558.1, AKG61631.1, AKG61705.1, AKG61776.1, AKG61849.1, AKG61922.1, AKG61995.1, AKH80465.1, AKH80538.1, ALM22637.1, ALM22711.1, ALM22785.1, ALM22859.1, ALO 18664.1, ALO 18740.1, AMB65664.1, AMB65737.1, AMB65811.1, AMB65887.1, AMB65958.1, AMN09834.1, ANN83966.1, ANN84043.1, ANN84119.1, ANN84196.1, ANN84273.1, ANN84350.1, ANN84426.1, ANN84502.1, ANN84579.1, ANN84655.1, ANN84732.1, ANN84808.1, ANN84885.1, ANN84961.1, ANN85038.1, ANN85114.1, ANN85189.1, ANN85266.1, ANN85343.1, ANN85418.1, ANN85496.1, ANN85573.1, ANN85650.1, ANN85726.1, ANN85803.1, AOY34085.1, AOY36687.1, ARB08959.1, AR038073.1, AR038074.1, AR038075.1, AR038076.1, AR038077.1, AR038078.1, AR038079.1, AR038080.1, ASM47642.1, ASM47666.1, ASM47743.1, ASM47820.1, ASM47895.1, BAM73421.1, CAA26062.1, CAA32272.1, CAF24756.1, CAF24757.1, CAF24758.1, CAF24759.1, CAF24760.1, CAF24761.1, CAF24762.1, CAF24763.1, CAF24764.1, CAF24765.1, CAF24766.1, CAF24767.1, CAF24768.1, CAF24769.1, CAF24770.1, CAF24771.1, CAF24772.1, CAF24773.1, CAF24774.1, CAF24775.1, CAF24776.1, CAF24777.1, CAF24778.1, CAF24779.1, CAF24780.1, CAF24781.1, CAF24782.1, CAF24783.1, CAF24784.1, CAF24785.1, P04290.1, P04488.1, P28986.1, Q703F0.1, SB007910.1, SBS69571.1, SBS69576.1, SBS69595.1, SBS69636.1, SBS69693.1, SBS69701.1, SBS69722.1, SBS69732.1, SBS69813.1, SBT69397.1, or YP_009137143.1.

In one embodiment, a gE fragment encoded by the mRNA used in the methods and compositions described herein comprises an IgG Fc-binding domain of the gE protein. In another embodiment, the gE domain encoded by mRNA is any other gE domain known in the art to mediate binding to IgG Fc.

In one embodiment, the immunogenic composition comprises an mRNA encoding HSV-2 gC. In another embodiment, the composition comprises an mRNA encoding HSV-2 gC set forth in SEQ ID NO: 13. A corresponding DNA sequence for in vitro preparation of this mRNA is shown in SEQ ID NO:14. In one embodiment, the immunogenic composition comprises an mRNA encoding the fragment of HSV-2 glycoprotein gC2 (amino acids 27-426) set forth in SEQ ID NO:15. In yet another embodiment, the immunogenic composition comprises an immunogenic fragment of HSV-2 gC, such as the extracellular domain region of HSV-2 gC (corresponding to amino acids 1-468 of SEQ ID NO:13).

In one embodiment, the HSV-1 gC encoded by the mRNA utilized in the methods and compositions described herein comprises the amino acid sequences as set forth in any one of the following GenBank Accession Numbers: AAA45779.1, AAA96680.1, ABI63505.1, ABM52973.1, ABM52976.1, ABM52977.1, ACM62267.1, ADD60042.1 ADD60119.1, ADM22367.1, ADM22444.1, ADM22520.1, ADM22597.1, ADM22674.1 ADM22751.1, ADM22827.1, ADM22904.1, ADM22981.1, ADM23057.1, ADM23133.1 ADM23210.1, ADM23287.1, ADM23361.1, ADM23435.1, ADM23509.1, ADM23583.1 ADM23658.1, ADM23733.1, ADM23809.1, AEQ77075.1, AEQ77099.1, AER37628.1, AER37697.1, AER37767.1, AER37838.1, AER37910.1, AER37981.1, AER38051.2 AFA36179.1, AFA36180.1, AFA36181.1, AFA36182.1, AFA36183.1, AFA36184.1 AFA36185.1, AFA36186.1, AFA36187.1, AFA36188.1, AFA36189.1, AFA36190.1 AFA36191.1, AFA36192.1, AFA36193.1, AFA36194.1, AFA36195.1, AFA36196.1 AFA36197.1, AFA36198.1, AFA36199.1, AFA36200.1, AFA36201.1, AFA36202.1 AFA36203.1, AFE62872.1, AFH78104.1, AFI23635.1, AFK50391.1, AFP86408.1, AGZ01906.1, AIR95840.1, AJE59989.1, AJE60060.1, AJE60131.1, AJE602021, AKE48623.1 AKE98415.1, AKE98416.1, AKE98417.1, AKE98418.1, AKE98419.1, AKE98420.1, AKE98421.1, AKE98422.1, AKE98423.1, AKE98424.1, AKE98425.1, AKE98426.1, AKE98427.1, AKE98428.1, AKE98429.1, AKE98430.1, AKE98431.1, AKE98432.1, AKE98433.1, AKE98434.1, AKE98435.1, AKG59227.1, AKG59299.1, AKG59372.1, AKG59444.1, AKG59516.1, AKG59591.1, AKG59663.1, AKG59736.1, AKG59807.1, AKG59879.1, AKG59953.1, AKG60027.1, AKG60099.1, AKG60170.1, AKG60243.1, AKG60316.1, AKG60386.1, AKG60456.1, AKG60528.1, AKG60601.1, AKG60674.1, AKG60745.1, AKG60817.1, AKG60887.1, AKG60959.1, AKG61032.1, AKG61104.1, AKG61175.1, AKG61248.1, AKG61321.1, AKG61392.1, AKG61464.1, AKG61537.1, AKG61611.1, AKG61684.1, AKG61756.1 AKG61828.1 AKG61902.1 AKG61974.1 AKH80444.1, AKH80517.1, AKM76368.1 ALM22613.1 ALM22687.1 ALM22761.1 ALM22835.1, AL018641.1, AL018717.1, AMB65642.1, AMB65715.1 AMB65862.1 AMN09813.1, ANN83942.1, ANN84019.1, ANN84095.1, ANN84172.1, ANN84249.1, ANN84326.1, ANN84403.1, ANN84478.1, ANN84555.1, ANN84632.1, ANN84708.1, ANN84785.1, ANN84861.1, ANN84938.1, ANN85014.1, ANN85091.1, ANN85167.1, ANN85242.1, ANN85319.1, ANN85396.1, ANN85472.1, ANN85549.1, ANN85626.1, ANN85703.1, ANN85779.1, AOY34308.1, AOY36663.1, AOY36687.1, ARB08935.1, ARO38059.1, AR038060.1, ARO38061.1, ARO38062.1, ARO38063.1, AR038064.1, ARO38065.1, ARO38066.1, ASM47642.1, ASM47719.1, ASM47796.1, ASM47871.1, BAM73394.1, CAA32294.1, CAB40083.1, CAD13356.1, CAD13357.1, CAD13358.1, CAD13359.1, CAD13360.1, CAD13361.1, CAD13362.1, CAD13363.1, CAD 13364.1, CAD13365.1, CAD13366.1, CAD13367.1, CAD13368.1, CAD13369.1, CAD 13370.1, CAD13371.1, CAD13372.1, CAD13373.1, CAD 13374.1, CAD13375.1, CAD 13376.1, CAD13377.1, CAD13378.1, P04290.1, P04488.1, P09855.1, P10228.1, P28986.1, SB007729.1 SB007793.1, SB007798.1, SB007812.1, SB007880.1, SBS69375.1, SBS69379.1, SBS69440.1 SBS69448.1, SBS69560.1, SBS69599.1, SBS69602.1, SBS69637.1, SBS69790.1, SBT69374.1 SCL76887.1, YP_009137119.1, or YP_009137143.1.

The glycoproteins, such as gC and gD, and fragments thereof can be those described in U.S. Pat. No. 9,284,355, which is hereby incorporated by reference.

The mRNA may be modified as described in International Publication No. WO 2019/035066, for example, by having one or more nucleosides in the mRNA comprise one or more pseudouridine residues.

The nucleotides, which may be incorporated into a polynucleotide, primary construct, or mRNA molecule, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked polynucleotides, primary constructs, or mmRNA molecules are expected to also reduce the innate immune response through weaker binding/activation of cellular innate immune molecules.

In specific embodiments, a modified nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein below.

The polynucleotides, primary constructs, and mmRNA of the invention can include a combination of modifications to the sugar, the nucleobase, and/or the internucleoside linkage.

In another embodiment, the purified preparation of RNA, oligoribonucleotide, or polyribonucleotide of the methods and compositions of the present invention comprises a combination of two or more of the above-described modifications. In another embodiment, the purified preparation of the RNA or ohgoribonucleotide comprises a combination of three or more of the above-described modifications. In another embodiment, the purified preparation of the RNA or ohgoribonucleotide comprises a combination of more than three of the above-described modifications.

In one embodiment, the modified mRNAs comprise in vitro-synthesized modified mRNAs.

In one embodiment, the mRNA comprises one or more modified mRNAs encoding an HSV glycoprotein. In one embodiment, the modified RNA comprises pseudouridine or pseudouridine family residues. In another embodiment, the modified mRNAs are capable of directing protein expression of HSV glycoproteins encoded thereon.

In another embodiment, mRNA provides an in vitro-transcribed mRNA molecule encoding an HSV glycoprotein, comprising a pseudouridine. In another embodiment, the mRNA is a synthetic mRNA molecule encoding an HSV glycoprotein, comprising a pseudouridine.

In another embodiment, an in vitro-transcribed mRNA molecule of the methods and compositions described herein is synthesized by T7 phage RNA polymerase. In another embodiment, the molecule is synthesized by SP6 phage RNA polymerase. In another embodiment, the molecule is synthesized by T3 phage RNA polymerase. In another embodiment, the molecule is synthesized by a polymerase selected from the above polymerases. In another embodiment, the mRNA is synthesized chemically on a column similar to DNA.

In another embodiment, the nucleoside that is modified in an RNA, ohgoribonucleotide, or polyribonucleotide of the methods and compositions described herein is uridine (U). In another embodiment, the modified nucleoside is cytidine (C). In another embodiment, the modified nucleoside is adenine (A). In another embodiment the modified nucleoside is guanine (G).

In another embodiment, the modified mRNA of the methods and compositions described herein further comprises a poly-A tail. In another embodiment, the modified mRNA of the methods and compositions described herein does not comprise a poly-A tail.

In another embodiment, the modified mRNA of the methods and compositions described herein comprises an m7GpppG cap. In another embodiment, the modified mRNA of the methods and compositions described herein does not comprise an m7GpppG cap. In another embodiment, the modified mRNA of the methods and compositions described herein comprises a 3′-O-methyl-m7GpppG. In another embodiment, the modified mRNA of methods and composition described herein comprise a non-reversible cap analog, which, in one embodiment, is added during transcription of the mRNA. In another embodiment, the modified mRNA of methods and composition described herein comprise an anti-reverse cap analog.

In another embodiment, the modified mRNA of the methods and compositions described herein further comprises a cap-independent translational enhancer. In another embodiment, the modified mRNA of the methods and compositions described herein does not comprise a cap-independent translational enhancer. In another embodiment, the cap-independent translational enhancer is a tobacco etch virus (TEV) cap-independent translational enhancer. In another embodiment, the cap-independent translational enhancer is any other cap-independent translational enhancer known in the art.

In another embodiment, between 0.1% and 100% of the uridine residues in the modified mRNAs of the methods and compositions described herein are modified (e.g. by the presence of pseudouridine). In another embodiment, the fraction of uridine residues modified is less than 5%, 3%, or 1%.

In another embodiment, the terms “ribonucleotide,” “oligoribonucleotide,” and polyribonucleotide refers to, in one embodiment, compounds comprising nucleotides in which the sugar moiety is ribose. In another embodiment, the term includes both RNA and RNA derivates in which the backbone is modified. Numerous RNA backbone modifications are known in the art and contemplated in the present invention. In one embodiment, modified RNA is a PNA (peptide nucleic acid). PNA contains peptide backbones and nucleotide bases and are able to bind, in another embodiment, to both DNA and RNA molecules. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. Each nucleic acid derivative represents a separate embodiment of the present invention.

Methods for production of nucleic acids having modified backbones are well known in the art, and are described, for example in U.S. Pat. Nos. 5,723,335 and 5,663,153 and PCT publication WO 95/26204.

The nucleic acid of interest can be purified by any method known in the art. In one embodiment, the nucleic acid of interest is purified using high-performance liquid chromatography (HPLC). In another embodiment, the nucleic acid of interest is purified by contacting the nucleic acid of interest with the bacterial enzyme RNase III. In other various embodiments, any method of nucleic acid purification that substantially reduces the immunogenicity of the nucleic acid preparation can be used. Non-limiting examples of purification methods that can be used with the compositions and methods of the invention liquid chromatography separation and enzyme digestion, each used alone or in any combination, simultaneously or in any order. Non-limiting examples of liquid chromatography separation include HPLC and fast protein liquid chromatography (FPLC). Materials useful in the HPLC and FPLC methods of the invention include, but are not limited to, cross-linked polystyrene/divinylbenzene (PS/DVB), PS/DVB-C18, PS/DVB-alkylated, Helix DNA columns (Varian), Eclipse dsDNA Analysis Columns (Agilent Technologies), Reverse-phase 5 (RPC-5) exchange material, DNAPac, ProSwift, and bio-inert UltiMate® 3000 Titanium columns (Dionex). Enzymes useful in the enzyme digestion methods of the invention include any enzyme able to digest any contaminant in a nucleic acid preparation of the invention, such as, for example a dsRNA contaminant, and include but are not limited to, RNase III, RNase VI, Dicer, and Chipper (see Fruscoloni et al., 2002, PNAS 100:1639).

In one embodiment, the amount of each mRNA in the composition ranges from about 10 mcg to 30 mcg, e.g., per dose.

### Adjuvants

In one embodiment, the immunogenic composition comprises an adjuvant, while in another embodiment, the vaccines do not comprise an adjuvant. “Adjuvant” refers, in another embodiment, to compounds that, when administered to an individual or tested in vitro, increase the immune response to an antigen in the individual or test system to which the antigen is administered. In another embodiment, an immune adjuvant enhances an immune response to an antigen that is weakly immunogenic when administered alone, i.e., inducing no or weak antibody titers or cell-mediated immune response. In another embodiment, the adjuvant increases antibody titers to the antigen. In another embodiment, the adjuvant lowers the dose of the antigen effective to achieve an immune response in the individual.

In one embodiment, the adjuvant is a CpG-containing nucleotide sequence (e.g., a CpG-containing oligonucleotide or a CpG-containing oligodeoxynucleotide (CpG ODN)). In another embodiment, the adjuvant is ODN 1826, which in one embodiment, is acquired from InvivoGen (San Diego, CA). In one embodiment, the CpG nucleotide molecule is 7909, which is 5′ TCGTCGTTTTGTCGTTTTGTCGTT (SEQ ID NO: 16). In another embodiment, the CpG nucleotide molecule is 2216, which is 5′ GGGGGACGATCGTCGGGGGG (SEQ ID NO: 17). In one embodiment, the composition includes 10 mcg to 8 mg of the CpG oligonucleotide.

In another embodiment, the adjuvant is an aluminum salt adjuvant. The aluminum salt adjuvant can be an alum-precipitated vaccine, an alum-adsorbed vaccine, hydrated alumina e.g. alumina hydrate, such as alumina trihydrate (ATH)), aluminum hydrate (e.g., aluminum trihydrate, alhydrogel, aluminum (III) hydroxide, amorphous alumina, trihydrated alumina, and trihydroxyaluminum. The dose of the aluminum salt adjuvant may range from If meg to 5 mg.

In another embodiment, the adjuvant is a Montanide ISA adjuvant or a trimer of complement component C3d. In another embodiment, the trimer is covalently linked to the protein immunogen. In another embodiment, the adjuvant is MF59, a granulocyte/macrophage colony-stimulating factor (GM-CSF) protein, a nucleotide molecule encoding GM-CSF, saponin QS21, monophosphoryl lipid A (MPL), SBAS2, an immune-stimulating cytokine, a quill glycoside, a bacterial mitogen, a bacterial toxin, or any combination of any of the foregoing.

### Formulation

The composition may further comprise a pharmaceutically acceptable carrier. In the composition, the mRNA can be complexed with polymeric or lipid components, encapsulated in liposomes, or in lipid nanoparticles (LNPs).

In one embodiment, a method of present invention further comprises mixing the modified mRNA with a transfection reagent prior to the step of contacting. In another embodiment, a method of present invention further comprises administering the mRNA together with the transfection reagent. In another embodiment, the transfection reagent is a cationic or ionizable lipid reagent.

In another embodiment, the transfection reagent is a lipid-based transfection reagent. In another embodiment, the transfection reagent is a protein-based transfection reagent. In another embodiment, the transfection reagent is a polyethyleneimine based transfection reagent. In another embodiment, the transfection reagent is calcium phosphate. In another embodiment, the transfection reagent is Lipofectin® or Lipofectamine®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity.

In another embodiment, liposomes are hollow spherical vesicles composed of lipids arranged in a similar fashion as those lipids which make up the cell membrane. They have, in another embodiment, an internal aqueous space for entrapping water soluble compounds and range in size from 0.05 to several microns in diameter. In another embodiment, liposomes can deliver RNA to cells in a biologically active form (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

In another embodiment, a mRNA described herein is encapsulated in a nanoparticle. Methods for nanoparticle packaging are well known in the art, and are described, for example, in Bose S, et al. (Role of Nucleolin in Human Parainfluenza Virus Type 3 Infection of Human Lung Epithelial Cells, J. Virol. 78: 8146, 2004); Dong Y et al. Poly(d,l-lactide-co-glycolide)/montmorillonite nanoparticles for oral delivery of anticancer drugs, Biomaterials 26:6068, 2005); Lobenberg R. et al. (Improved body distribution of 14C-labelled AZT bound to nanoparticles in rats determined by radio luminography, J Drug Target 5:171, 1998); Sakuma S R et al (Mucoadhesion of polystyrene nanoparticles having surface hydrophilic polymeric chains in the gastrointestinal tract, Int J Pharm 177:161, 1999); Virovic L et al., Novel delivery methods for treatment of viral hepatitis: an update, Expert Opin Drug Deliv 2:707.2005); and Zimmermann E et al., Electrolyte- and pH-stabilities of aqueous solid lipid nanoparticle (SLN) dispersions in artificial gastrointestinal media, Eur J Pharm Biopharm 52:203. 2001).

In one embodiment, the mRNA is encapsulated in nanoparticles to improve efficiency of delivery and expression of the mRNA. Nanoparticle packaging involves condensing and encapsulating RNA into particles that are smaller than the pore of the nuclear membrane, using chemicals including poly-L-lysine and polyethylene glycol. In one embodiment, RNA is packaged into one of four nanoparticle formulations (PEI, PLL, PAE, and CK3oPEGiok).

In one embodiment, nanoparticles used in the compositions and methods described herein comprise lipid nanoparticles as described in Cullis, P., & Hope, M. (n.d.). Lipid Nanoparticle Systems for Enabling Gene Therapies, Molecular therapy, 25(7), which is incorporated by reference herein in its entirety.

In one embodiment, delivery of nucleoside-modified RNA comprises any suitable delivery method, including exemplary RNA transfection methods described elsewhere herein. In certain embodiments, delivery of a nucleoside-modified RNA to a subject comprises mixing the nucleoside-modified RNA with a transfection reagent prior to the step of contacting. In another embodiment, a method of present invention further comprises administering nucleoside-modified RNA together with the transfection reagent. In another embodiment, the transfection reagent is a cationic lipid reagent.

In another embodiment, the transfection reagent is a lipid-based transfection reagent. In another embodiment, the transfection reagent is a protein-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome.

Liposomes, in another embodiment, increase intracellular stability, increase uptake efficiency and improve biological activity. In another embodiment, liposomes are hollow spherical vesicles composed of lipids arranged in a similar fashion as those lipids which make up the cell membrane. They have, in another embodiment, an internal aqueous space for entrapping water-soluble compounds and range in size from 0.05 to several microns in diameter. In another embodiment, liposomes can deliver RNA to cells in a biologically active form.

In one embodiment, the composition comprises a lipid nanoparticle (LNP) and one or more nucleic acid molecules described herein. For example, in one embodiment, the composition comprises an LNP and one or more mRNA, and optionally one or more adjuvants.

The term “lipid nanoparticle” refers to a particle having at least one dimension on the order of nanometers (e.g., 1-1,000 nm) which includes one or more lipids, for example, a lipid as described in WO2016/176330, which is incorporated by reference herein in its entirety.

In some embodiments, lipid nanoparticles are included in a formulation comprising a nucleoside-modified RNA as described herein. In some embodiments, such lipid nanoparticles comprise a cationic lipid and one or more excipient selected from neutral lipids, charged lipids, steroids and polymer conjugated lipids (e.g., a pegylated lipid such as a pegylated lipid). In some embodiments, the nucleoside-modified RNA is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, the nucleoside-modified RNA, when present in the lipid nanoparticles, is resistant in aqueous solution to degradation with a nuclease.

The LNP may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glyco lipids; and (3) “derived lipids” such as steroids.

In one embodiment, the LNP comprises one or more cationic lipids, and one or more stabilizing lipids. Stabilizing lipids include neutral lipids and pegylated lipids.

In one embodiment, the LNP comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In certain embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Choi), N-(1-(2,3-dioleoyloxy)propyl)-N-2-(sperminecarboxamido)ethyl)-N,N-dimethylammonium trifluoracetate (DOSPA), dioctadecylamidoglycyl carboxy spermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt (DLin-TAP·Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In certain embodiments, the cationic lipid is present in the LNP in an amount from about 30 to about 95 mole percent. In one embodiment, the cationic lipid is present in the LNP in an amount from about 30 to about 70 mole percent. In one embodiment, the cationic lipid is present in the LNP in an amount from about 40 to about 60 mole percent. In one embodiment, the cationic lipid is present in the LNP in an amount of about 50 mole percent. In one embodiment, the LNP comprises only cationic lipids. In certain embodiments, the L P comprises one or more additional lipids which stabilize the formation of particles during their formation.

Suitable stabilizing lipids include neutral lipids and anionic lipids.

The term “neutral lipid” refers to any one of a number of lipid species that exist in either an uncharged or neutral zwitterionic form at physiological pH.

Representative neutral lipids include diacylphosphatidylcho lines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-mono methyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In various embodiments, the LNPs further comprise a steroid or steroid analogue.

In certain embodiments, the steroid or steroid analogue is cholesterol. In some of these embodiments, the molar ratio of the cationic lipid to cholesterol ranges from about 2:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In certain embodiments, the LNP comprises glycolipids (e.g., monosialoganglioside GMi). In certain embodiments, the LNP comprises a sterol, such as cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG).

In certain embodiments, the LNP comprises an additional, stabilizing lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols.

Representative polyethylene glycol-lipids include PEG-c-DOMG, PEG-c-DMA, and PEG-s-DMG. In one embodiment, the polyethylene glycol-lipid is N-[(methoxy poly(ethylene glycol)20oo)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(co-methoxy(polyethoxy)ethyl)butanedioate (PEG-S-DMG), a pegylated ceramide (PEG-cer), or a PEG dialkoxypropylcarbamate such as Q-methoxy(polyethoxy)ethyl-N-(2,3-di(tetradecanoxy)propyl)carbamate or 2,3-di(tetradecanoxy)propyl-N-(co-methoxy(polyethoxy)ethyl)carbamate. In various embodiments, the molar ratio of the cationic lipid to the pegylated lipid ranges from about 100:1 to about 25:1.

In certain embodiments, the additional lipid is present in the LNP in an amount from about 1 to about 10 mole percent. In one embodiment, the additional lipid is present in the LNP in an amount from about 1 to about 5 mole percent. In one embodiment, the additional lipid is present in the LNP in about 1 mole percent or about 1.5 mole percent.

In certain embodiments, the LNP comprises one or more targeting moieties which are capable of targeting the LNP to a cell or cell population. For example, in one embodiment, the targeting moiety is a ligand which directs the LNP to a receptor found on a cell surface.

In certain embodiments, the LNP comprises one or more internalization domains. For example, in one embodiment, the LNP comprises one or more domains which bind to a cell to induce the internalization of the LNP. For example, in one embodiment, the one or more internalization domains bind to a receptor found on a cell surface to induce receptor-mediated uptake of the LNP. In certain embodiments, the LNP is capable of binding a biomolecule in vivo, where the LNP-bound biomolecule can then be recognized by a cell-surface receptor to induce internalization. For example, in one embodiment, the LNP binds systemic ApoE, which leads to the uptake of the LNP and associated cargo.

Other exemplary LNPs and their manufacture are described in the art, for example in WO2016/176330, U.S. Patent Application Publication No. US2012/0276209, Semple et al., 2010, Nat Biotechnol., 28(2): 172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5):E781-90; Belliveau et al., 2012, Mol Ther Nucleic Acids, 1:e37; Jayaraman et al., 2012, Angew Chem Int Ed Engl., 51(34):8529-8533; Mui et al., 2013, Mol Ther Nucleic Acids, 2, e139; Maier et al., 2013, Mol Ther., 21(8):1570-1578; and Tarn et al., 2013, Nanomedicine, 9(5):665-74, each of which are incorporated by reference in their entirety.

### Methods of Treatment

Yet another embodiment is a method of inducing an anti-HSV immune response in a mammalian subject (e.g., a human subject) by administering to the subject an effective amount of the immunogenic composition described herein. The method may further comprise the step of administering to the subject a booster vaccination which comprises the immunogenic composition described herein.

Yet another embodiment is a method of suppressing, inhibiting, or reducing an incidence of an HSV infection (e.g., an HSV-1 infection or HSV-2 infection) in a mammalian subject (e.g., a human subject) by administering to the subject an effective amount of the immunogenic composition described herein. In one embodiment, the HSV infection is an HSV-1 infection. In one embodiment, the HSV infection is an HSV-2 infection. In one embodiment, the HSV infection is a primary HSV infection. In one embodiment, the HSV infection is a flare, recurrence, or HSV labialis or genital herpes following a primary HSV infection. In one embodiment, the HSV infection is HSV encephalitis. In one embodiment, the HSV infection is HSV keratisis or conjuctivitis. In one embodiment, the HSV infection is an HSV neonatal infection. In one embodiment, the HSV infection is an HSV eye infection. In one embodiment, the subject is HIV-infected.

Yet another embodiment is a method of inducing an anti-HSV immune response in a mammalian subject comprising providing to the subject (a) mRNA encoding herpesvirus glycoprotein D or an immunogenic fragment thereof, (b) mRNA encoding herpesvirus glycoprotein B or an immunogenic fragment thereof, and (c) optionally an adjuvant, where the mRNA for herpesvirus glycoprotein B is translated to glycoprotein B in prefusion fixed form. In one embodiment, the method further comprises providing to the subject mRNA encoding herpesvirus glycoprotein C or an immunogenic fragment thereof, herpesvirus glycoprotein E or an immunogenic fragment thereof, or a combination thereof. In another embodiment, the subject is not administered mRNA encoding herpesvirus glycoprotein H or an immunogenic fragment thereof or mRNA encoding herpesvirus glycoprotein L or an immunogenic fragment thereof. In yet embodiment, the subject is not administered (i) mRNA encoding herpesvirus glycoprotein H or an immunogenic fragment thereof, (ii) herpesvirus glycoprotein H or an immunogenic fragment thereof, (iii) mRNA encoding herpesvirus glycoprotein L or an immunogenic fragment thereof, or (iv) herpesvirus glycoprotein L or an immunogenic fragment thereof.

Yet another embodiment is a method of vaccinating a mammalian subject against herpes simplex virus (such as HSV-1 or HSV-2) comprising providing to the mammalian subject (e.g., a human subject) mRNA encoding herpesvirus glycoprotein D or an immunogenic fragment thereof and herpesvirus glycoprotein B or an immunogenic fragment thereof, where the mRNA for herpesvirus glycoprotein B is translated to glycoprotein B in prefusion fixed form. In one embodiment, the method further comprises providing mRNA encoding herpesvirus glycoprotein C or an immunogenic fragment thereof, herpesvirus glycoprotein E or an immunogenic fragment thereof, or a combination thereof. In one embodiment, the subject is not administered mRNA encoding herpesvirus glycoprotein H or an immunogenic fragment thereof or herpesvirus glycoprotein L or an immunogenic fragment thereof.

The mRNA may be provided to the subject by administration of an immunogenic or vaccine composition comprising the mRNA, for example, as described herein. In one embodiment, the composition does not comprise mRNA encoding herpesvirus glycoprotein H or an immunogenic fragment thereof or herpesvirus glycoprotein L or an immunogenic fragment thereof. In another embodiment, the composition does not comprise (i) mRNA encoding herpesvirus glycoprotein H or an immunogenic fragment thereof, (ii) herpesvirus glycoprotein H or an immunogenic fragment thereof, (iii) mRNA encoding herpesvirus glycoprotein L or an immunogenic fragment thereof, or (iv) herpesvirus glycoprotein L or an immunogenic fragment thereof. In one embodiment, the composition is administered to the subject at least twice.

In any of the methods described herein, the mRNA may be administered systemically, intramuscularly, intradermally, subcutaneously, intravaginally, or parenterally or by intracerebroventricular or intraperitoneal injection.

One embodiment is a method of inhibiting spread of HSV by administering an immunogenic composition described herein. In one embodiment, the spread from DRG to skin is inhibited. In one embodiment, cell-to-cell spread of HSV is inhibited. In one embodiment, anterograde spread is inhibited. In one embodiment, retrograde spread is inhibited. “DRG” refers, in one embodiment, to a neuronal cell body and in another embodiment, contain the neuron cell bodies of nerve fibers. In another embodiment, the term refers to any other definition of “DRG” used in the art, such as dorsal root ganglia. In another embodiment, spread of HSV to neural tissue is inhibited.

Yet another embodiment is a method of inhibiting or preventing a recurrence following an HSV infection (such as a primary HSV infection) in a subject by administering to the subject an immunogenic composition (such as a vaccine composition) described herein. Yet another embodiment is a method of preventing a recurrence following a primary HSV infection in a subject by administering to the subject an immunogenic composition (such as a vaccine composition) described herein.

Yet another embodiment is a method of inhibiting an HSV labialis following a primary HSV infection in a subject by administering to the subject an immunogenic composition (such as a vaccine composition) described herein.

Yet another embodiment is a method of diminishing the severity of a recurrence of an HSV infection in a subject by administering to the subject an immunogenic composition (such as a vaccine composition) described herein. Yet another embodiment is a method of reducing the frequency of a recurrence of an HSV infection in a subject by administering to the subject an immunogenic composition (such as a vaccine composition) described herein. In one embodiment, the subject in any of the methods described herein is an HIV-infected subject.

Yet another embodiment is a method of treating, or reducing an incidence of, an HSV encephalitis in a subject by administering to the subject an immunogenic composition (such as a vaccine composition) described herein. “HSV encephalitis” refers, in one embodiment, to an encephalitis caused by a Herpes Simplex Virus-1 (HSV). In another embodiment, the term refers to an encephalitis associated with HSV. In another embodiment, the term refers to any other type of HSV-mediated encephalitis known in the art.

Yet another embodiment is a method of treating or reducing an HSV neonatal infection in a subject by administering to the subject an immunogenic composition (such as a vaccine composition) described herein.

Yet another embodiment is a method of treating, or reducing an incidence of, an HSV-mediated herpetic ocular disease in a subject by administering to the subject an immunogenic composition (such as a vaccine composition) described herein. Yet another embodiment is a method of treating, or reducing an incidence of, an HSV-1 corneal infection or herpes keratitis in a subject by administering to the subject an immunogenic composition (such as a vaccine composition) described herein.

Yet another embodiment is a method of treating, suppressing or inhibiting an HSV genital infection or any manifestation of recurrent HSV infection by administering to the subject an immunogenic composition (such as a vaccine composition) described herein.

Yet another embodiment is a method of reducing an incidence of an HSV-mediated genital ulcer disease in a subject by administering to the subject an immunogenic composition (such as a vaccine composition) described herein.

Yet another embodiment is a method of impeding an establishment of a latent HSV infection in a subject by administering to the subject an immunogenic composition (such as a vaccine composition) described herein.

Yet another embodiment is a method of reducing an incidence of an HSV-mediated encephalitis in a subject by administering to the subject an immunogenic composition (such as a vaccine composition) described herein. The herpes-mediated encephalitis ray be a focal herpes encephalitis or neonatal herpes encephalitis.

Yet another embodiment is a method of treating or reducing an incidence of a disease, disorder, or symptom associated with or secondary to a HSV-mediated encephalitis in a subject by administering to the subject an immunogenic composition (such as a vaccine composition) described herein.

Yet another embodiment is a method of treating, reducing the pathogenesis of, ameliorating the symptoms of, ameliorating the secondary symptoms of, reducing the incidence of, prolonging the latency to a relapse of a Herpes Simplex Virus (HSV) infection in a subject by administering to the subject an immunogenic composition (such as a vaccine composition) described herein.

Yet another embodiment is a method of protecting a subject against (or inhibiting the) formation of a zosteriform lesion or an analogous outbreak in a human subject by administering to the subject an immunogenic composition (such as a vaccine composition) described herein. The term “zosteriform” refers, in one embodiment, to skin lesions characteristic of an HSV infection, particularly during reactivation infection, which, in one embodiment, begin as a rash and follow a distribution near dermatomes, commonly occurring in a strip or belt-like pattern.

Yet another embodiment is a method of impeding the formation of a dermatome lesion or an analogous condition in a subject by administering to the subject an immunogenic composition (such as a vaccine composition) described herein. In one embodiment, dermatome lesions form as a result of contact with HSV.

It is to be understood that the methods of the present invention may be used to treat, inhibit, or suppress an HSV infection or primary or secondary symptoms related to such an infection following exposure of the subject to HSV. In another embodiment, the subject has been infected with HSV before vaccination. In another embodiment, the subject is at risk for HSV infection.

In one embodiment, “treating” refers to either therapeutic treatment or prophylactic or preventative measures, wherein the object is to prevent or lessen the targeted pathologic condition or disorder as described hereinabove. Thus, in one embodiment, treating may include directly affecting or curing, suppressing, inhibiting preventing, reducing the severity of, delaying the onset of, reducing symptoms associated with the disease, disorder or condition, or a combination thereof. Thus, in one embodiment., “treating” refers inter alia to delaying progression, expediting remission inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof

In one embodiment, symptoms are primary while in another embodiment, symptoms are secondary. In one embodiment, “primary” refers to a symptom that is a direct result of the subject viral infection, while in one embodiment, “secondary” refers to a symptom that is derived from or consequent to a primary cause. In one embodiment, the compositions and strains for use in the present invention treat primary or secondary symptoms or secondary complications related to HSV infection.

In another embodiment, “symptoms” may be any manifestation of HSV infection, comprising blisters, ulcerations, or lesions on the urethra, cervix, upper thigh, and/or anus in women and on the penis, urethra, scrotum, upper thigh and anus in men, inflammation, swelling, fever; flu-like symptoms sore mouth, sore throat; pharyngitis, pain, blisters on tongue, mouth or lips, ulcers, cold sores, neck pain, enlarged lymph nodes, reddening, bleeding, itching, dysuria, headache, muscle pain, or a combination thereof.

In another embodiment, the disease, disorder, or symptom is fever. In another embodiment, the disease, disorder, or symptom is headache. In another embodiment, the disease, disorder, or symptom is stiff neck. In another embodiment, the disease, disorder, or symptom is seizures. In another embodiment, the disease, disorder, or symptom is partial paralysis. In another embodiment the disease, disorder, or symptom is stupor. In another embodiment, the disease, disorder, or symptom is coma. In another embodiment, the disease, disorder, or symptom is any other disease, disorder, or symptom known in the art that is associated with or secondary to a herpes-mediated encephalitis.

### EXAMPLES

The following examples are included to demonstrate aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

mRNA was prepared to encode gC2 amino acids 27 to 426 (SEQ ID NO:15), gD2 amino acids 26 to 331 (SEQ ID NO:9), and gE2 amino acids 24 to 405 (SEQ ID NO:12), each truncated before their transmembrane domains. Balb/c mice were intramuscularly immunized with mixtures of the mRNA. The mice were then intravaginally challenged with 2×10⁶ plaque-forming units (pfu) of HSV-2 containing the luciferase gene to assess protection efficiency by the vaccinations.

There were eight variations of mRNA vaccine compositions tested using in vivo vaccination, each with a different combination of HSV surface glycoproteins: (1) gD, gB, gE, gC; (2) gB, gE, gC; (3) gD, gE, gC; (4) gD, gB, gH, gE, gC; (5) gD, gB-pf, gE, gC; (6) gB-pf, gE, gC; (7) Luc mRNA; and (8) HSV-2 (FusOn-H3, 2×10⁶) followed by mRNA vaccine (gD, gB, gH, gE, gC). Each composition was tested in six Balb/c mice.

Blood was collected pre-vacination. The vaccine compositions were administered twice with three weeks between administration. Two weeks after vaccination was completed, blood was collected. The mice where then challenged by administering progesterone one day after blood collection, and three days thereafter inoculation with 2×10⁶ pfu in 10 μl of HSV-2 strain 186 containing the luciferase gene (HSV-2-luc). The vaccine dosage was 10 μg per combinatorial immunogen. IVIS imaging was performed on the mice on Day 2, 4, and 6 after HSV-2 inoculation. The mice were clinically observed and their body weight monitored daily for two weeks. Vaginal fluid collection was performed on the mice on Day 1, 3, 5, and 7 by swap.

The results of the vaginal swap samples on Day 1, 3, 5 and 7 are shown in qPCR graphs analyzing DNA copies and log 10 DNA copies of expressed HSV glycoproteins in mice inoculated with the various mRNA vaccine compositions.

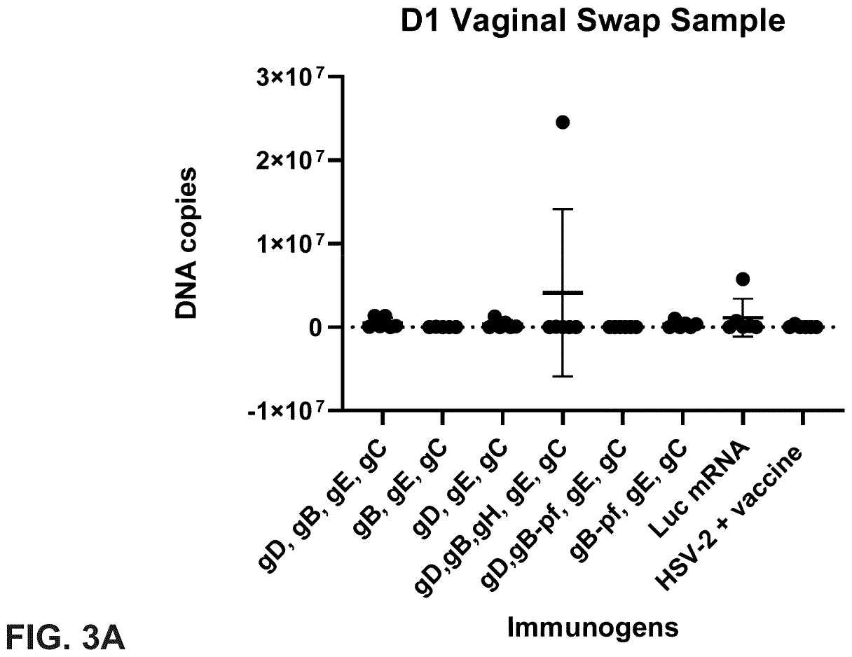

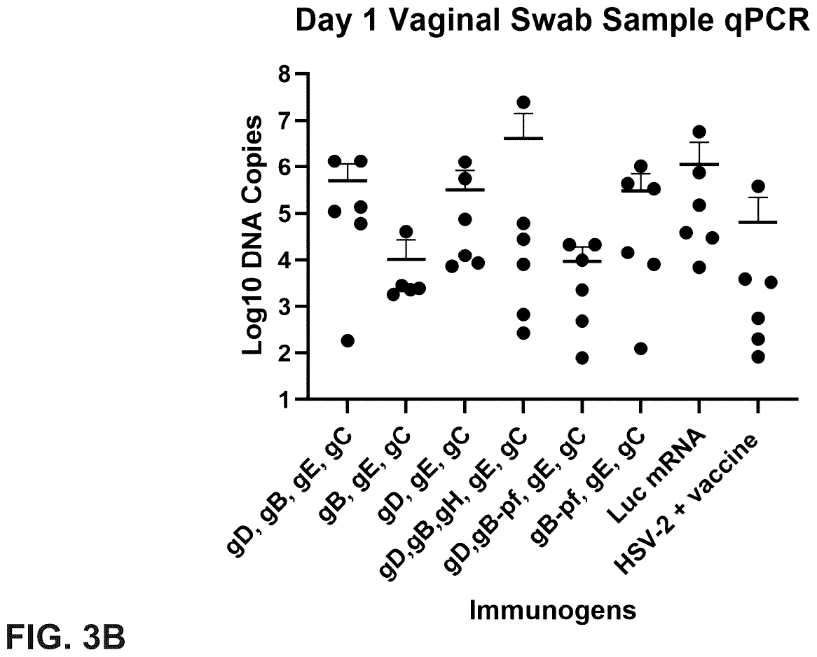

FIGS. 3A, 4A, 5A, and 6A are graphs showing qPCR results of day 1 (D1), day 3 (D3), day 5 (D5), and day 7 (D7) vaginal swabs, measuring DNA copies of HSV-2

FIGS. 3B, 4B, 5B, and 6B are graphs showing qPCR results of day 1 (D1), day 3 (D3), day 5 (D5), and day 7 (D7) vaginal swabs, logarithmically measuring DNA copies of HSV-2.

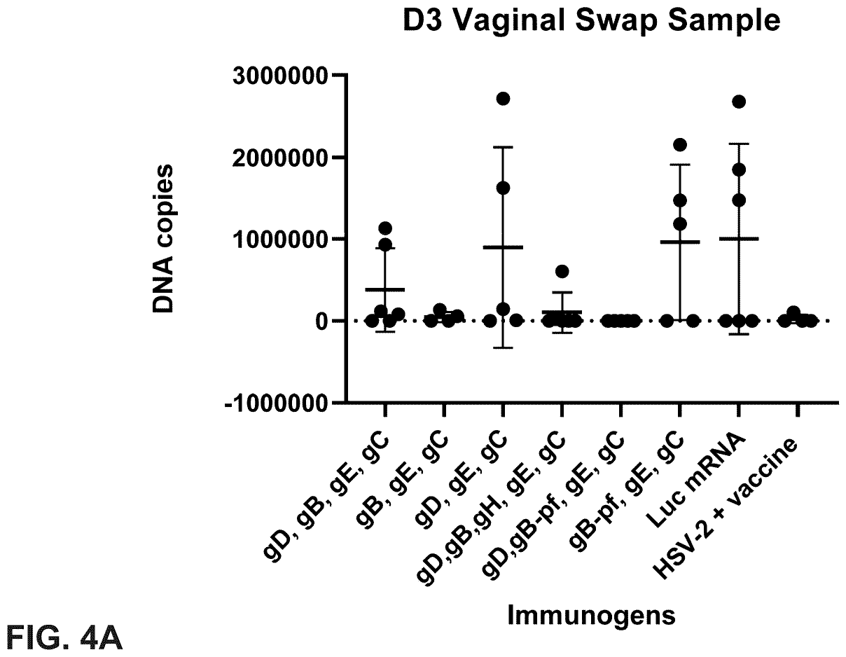

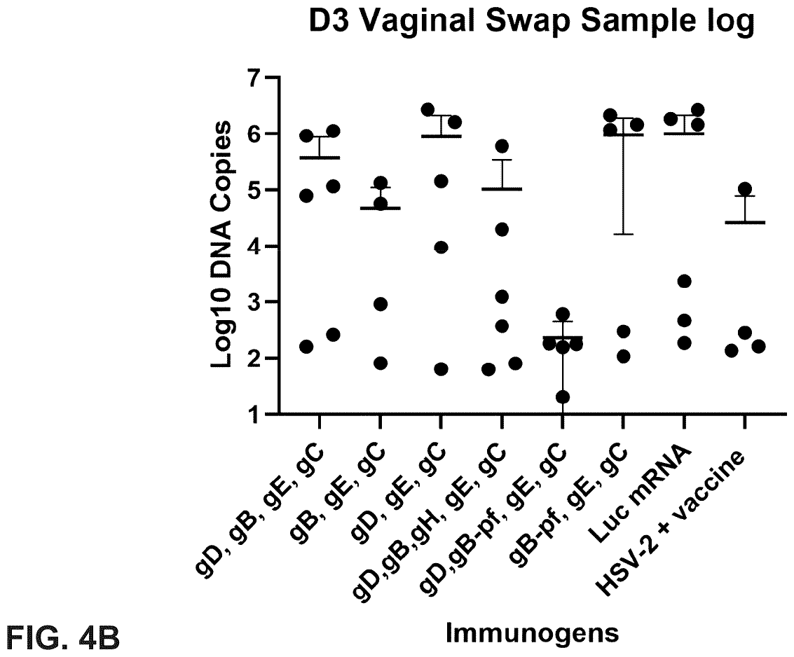

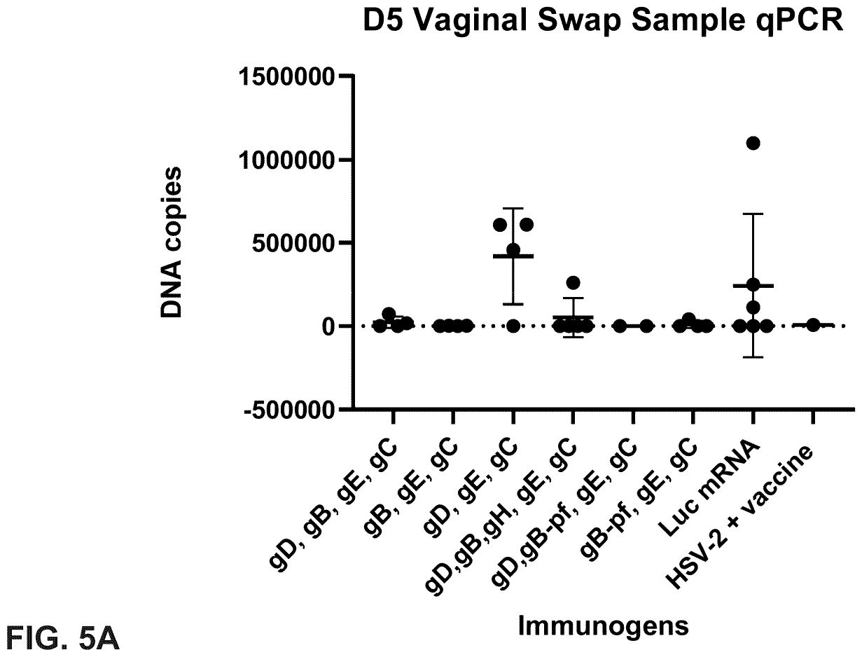

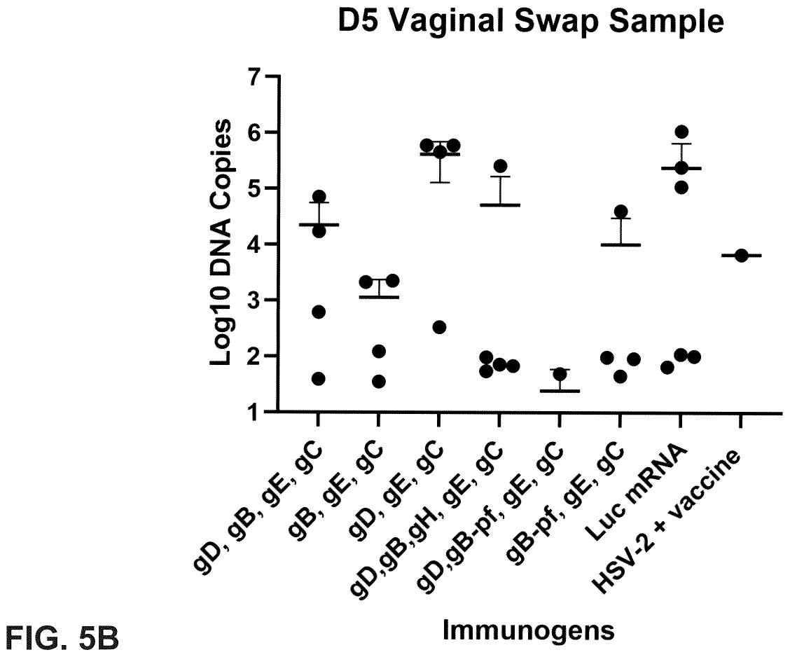

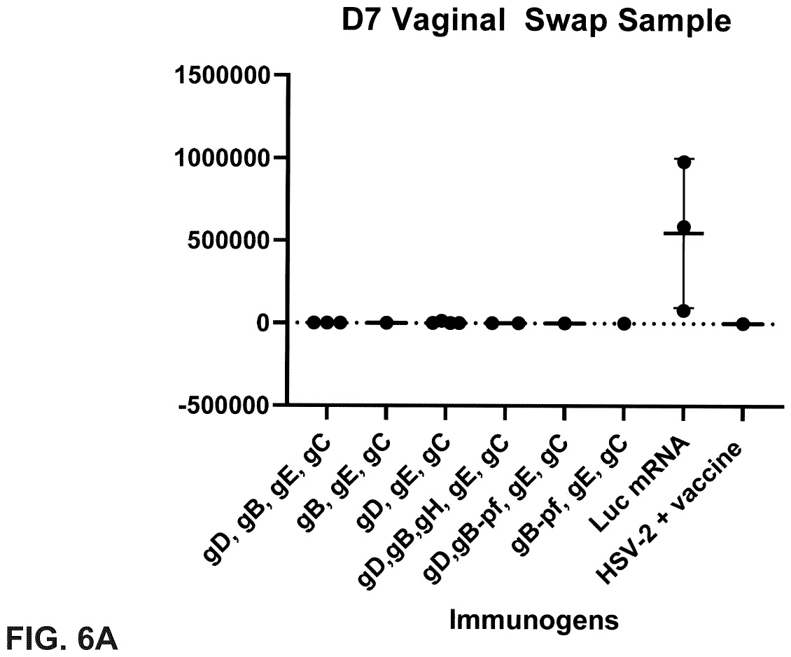

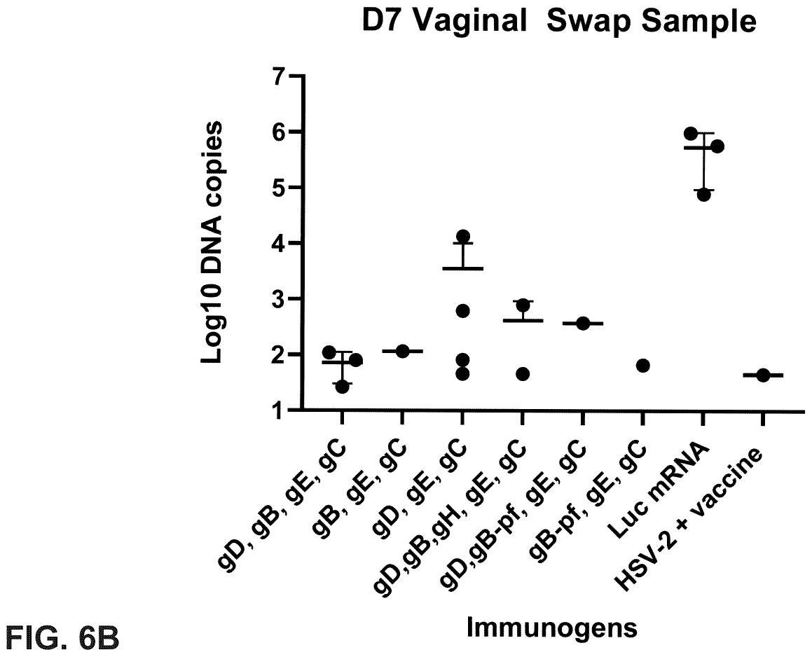

As shown in the graphs, while it is difficult to observe an initial difference in DNA copies of the glycoproteins (due to the remaining input viruses), by Day 3 (FIG. 4B) when the input virus was mostly gone and all the viruses were the newly produced from infection, it is apparent that the composition that is comprised of a combination of gD and gB-pf mRNA suppresses DNA expression of viral glycoproteins more effectively than other compositions, including those that combine gD with gB. Subsequent results on Day 5 (FIG. 5B) and Day 7 (FIG. 6B) confirm those findings.

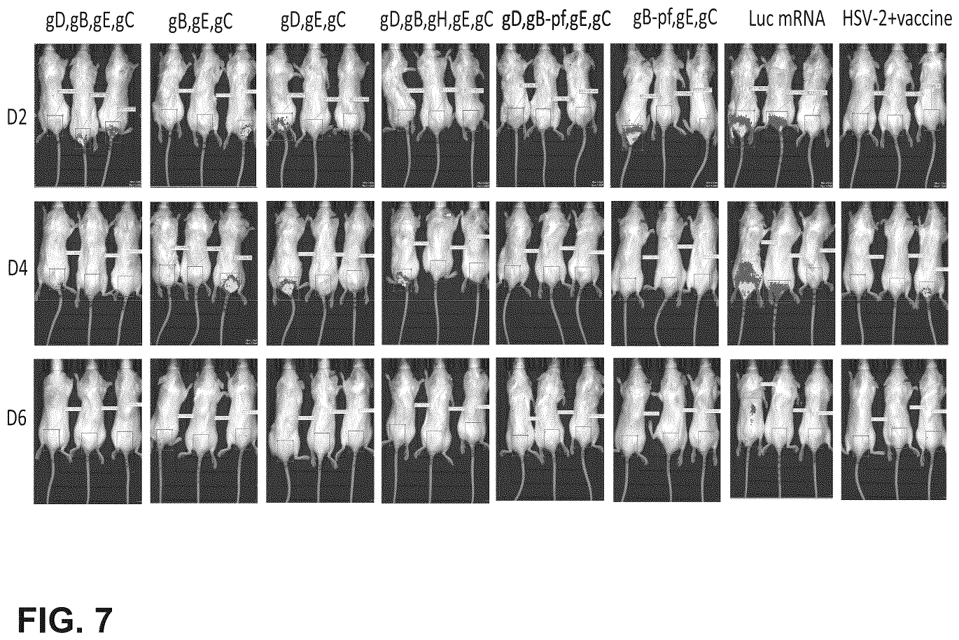

FIG. 7 shows IVIS in vivo images of mice inoculated with mRNA vaccines at day 2 (D2), day 4 (D4), and (D6), showing HSV-2-lu infection in tissue. The images demonstrate that there is significantly decreased viral activity in mice inoculated with the vaccine composition comprised of mRNA for gD, gB-pf, gE, and gC, as there is single mouse showing luciferase detection.

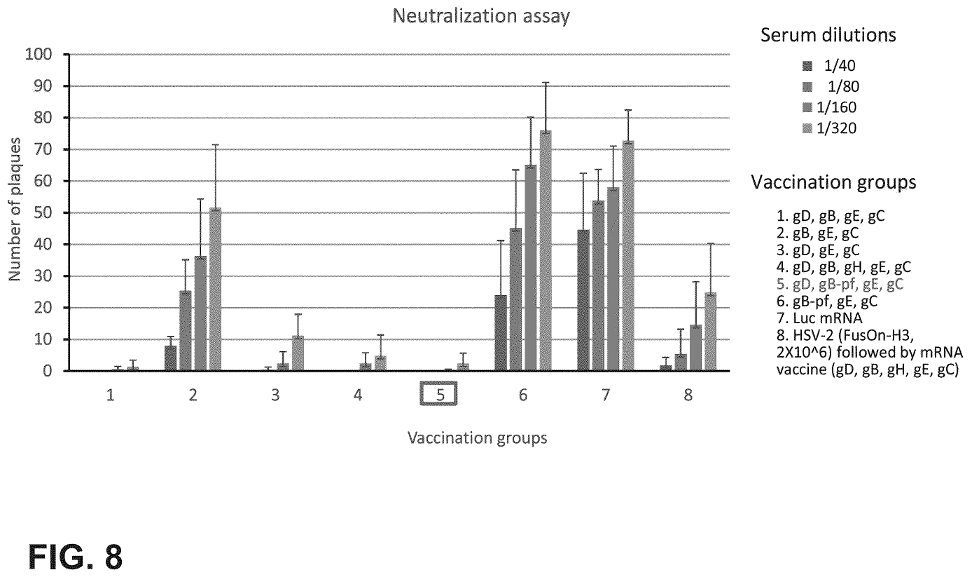

FIG. 8 shows the neutralization effect of blood collected at two weeks after the vaccination was completed on the infection of Vero cells in vitro. The blood sera were first diluted at 1:40, 1:80, 1:160, and 1:320. The diluted sera were then mixed with 100 pfu of HSV-2. After 1 hour incubation, the mixed samples were added to Vero cell monolayers for virus plaque formation to determine the inhibitory effect of the sera on virus infectivity. The result shows that sera from mice inoculated with the vaccine composition comprised of mRNA for gD, gB-pf, gE, and gC completely inhibited virus infectivity at dilutions 1:40, 1:80 and 1:160. Even at dilutions as high as 1:320, the virus infectivity was still vastly neutralized.

The sequences described herein are provided in the table below.

| Description | Sequence |
|---|---|
| HSV-2 gB-pf | MRGGGLICALVVGALVAAVASAAPAAPAAPRASGGVAATVAANGGP |
| (aa sequence) | ASRPPPVPSPATTKARKRKTKKPPKRPEATPPPDANATVAAGHATV |
| (SEQ ID NO: 1) | RAHLREIKVENADAQFYVCPPPTGATVVQFEQPRRCPTRPEGQNYT |
| | EGIAVVFKENIAPYKFKATMYYKDVTVSQVWFGHRYSQFMGIFEDR |
| | APVPFEEVIDKINAKGVCRSTAKYVRNNMETTAFHRDDHETDMELK |
| | PAKVATRTSRGWHTTDLKYNPSRVEAFHRYGTTVNCIVEEVDARSV |
| | YPYDEFVLATGDFVYMSPFYGYREGSHTEHTTYAADRFKQVDGFYA |
| | RDLTTKARATSPTTRNLLTTPKFTVAWDWVPKRPAVCTMTKWQEVD |
| | EMLRAEYGGSFRFSSDAISTTFTTNLTEYSLSRVDLGDCIGRDARE |
| | AIDRMFARKYNATHIKVGQPQYYQATGGFLIAYQPLLSNTLAELYV |
| | REYMREQDRKPRNATPAPLREAPSANASVERIKTTSSIEFARLQFT |
| | YNHIQRHVNDMLGRIAVAWCELQNHELTLWNEARKLNPNAIASATV |
| | GRRVSARMLGDVMAVATCVPVAPDNVIVQNSMRVSSRPGTCYSRPL |
| | VSFRYEDQGPLIEGQLGENNDVRLTRDALEPCTVGHRGYFIFGGGY |
| | VYFEEYAYSHQLSRADVTTVSTFIDLNITMLEDHEFVPLEVYTRHE |
| | IKDSGLLDYTEVQRRNQLHDLRFADIDTVIRADANAAMFAGLCAFF |
| | EGMGDLGRAVGKVVMGVVGGVVSAVSGVSSFMSNPFGALAVGLLVL |
| | AGLVAAFFAFRYVLQLQRNPMKALYPLTTKELKTSDPGGVGGEGEE |
| | GAEGGGFDEAKLAEAREMIRYMALVSAMERTEHKARKKGTSALLSS |
| | KVTNMVLRKRNKARYSPLHNEDEAGDEDEL |
| HSV-2 gB-pf | ATGAGGGGCGGCGGCCTGATCTGCGCCCTGGTGGTGGGCGCCCTGG |
| (DNA | TGGCCGCCGTGGCCAGCGCCGCACCAGCAGCACCAGCAGCCCCCAG |
| sequence) | GGCCTCCGGCGGCGTGGCCGCCACCGTGGCCGCCAACGGCGGCCCA |
| (SEQ ID NO: 2) | GCCTCCCGCCCCCCACCAGTGCCAAGCCCCGCCACCACAAAGGCCA |
| | GGAAGCGCAAGACAAAGAAGCCACCCAAGAGGCCAGAGGCAACCCC |
| | ACCCCCAGACGCAAACGCAACCGTGGCAGCAGGACACGCCACAGTG |
| | AGGGCCCACCTGCGCGAGATCAAGGTGGAGAACGCCGACGCCCAGT |
| | TCTACGTGTGCCCACCACCCACCGGAGCAACAGTGGTGCAGTTCGA |
| | GCAGCCACGGAGATGCCCAACCAGGCCAGAGGGCCAGAACTACACA |
| | GAGGGCATCGCCGTGGTGTTCAAGGAGAACATCGCCCCATACAAGT |
| | TCAAGGCCACCATGTACTACAAGGACGTGACAGTGAGCCAAGTGTG |
| | GTTCGGCCACCGCTACAGCCAGTTCATGGGCATCTTCGAGGACCGG |
| | GCCCCTGTGCCCTTCGAGGAAGTGATCGACAAGATCAACGCCAAGG |
| | GCGTGTGCAGGAGCACCGCCAAGTACGTGCGCAACAACATGGAGAC |
| | CACCGCCTTCCACAGGGACGACCACGAGACCGACATGGAGCTGAAG |
| | CCAGCCAAGGTGGCCACCAGGACCTCCCGCGGCTGGCACACCACCG |
| | ACCTGAAGTACAACCCCAGCCGGGTGGAGGCCTTCCACAGATATGG |
| | CACCACCGTGAACTGCATCGTGGAGGAGGTGGATGCCCGCTCCGTG |
| | TACCCCTACGACGAGTTCGTGCTGGCCACCGGCGACTTCGTGTACA |
| | TGAGCCCCTTCTACGGCTACCGGGAGGGCAGCCACACCGAGCACAC |
| | CACCTACGCCGCCGACAGGTTCAAGCAGGTGGACGGCTTCTACGCC |
| | CGGGACCTGACCACCAAGGCCAGGGCCACCAGCCCCACCACCAGGA |
| | ACCTGCTGACCACCCCAAAGTTCACCGTGGCCTGGGACTGGGTGCC |
| | CAAGAGGCCAGCCGTGTGCACCATGACCAAGTGGCAGGAGGTGGAC |
| | GAGATGCTGAGGGCCGAGTACGGCGGCTCCTTCAGGTTCAGCAGCG |
| | ACGCCATCAGCACCACCTTCACCACCAACCTGACCGAGTACAGCCT |
| | GAGCCGGGTGGACCTGGGCGACTGCATCGGAAGGGACGCCAGAGAG |
| | GCAATCGACCGGATGTTCGCCAGAAAGTACAACGCCACACACATCA |
| | AGGTCGGCCAGCCCCAGTACTACCAGGCCACCGGCGGCTTCCTGAT |
| | CGCCTACCAGCCCCTGCTGAGCAACACCCTGGCCGAGCTGTACGTG |
| | AGGGAGTACATGCGCGAGCAGGACAGGAAGCCCAGGAACGCCACCC |
| | CAGCACCCCTGAGGGAGGCACCCTCCGCCAACGCCAGCGTGGAGAG |
| | GATCAAGACCACCTCCAGCATCGAGTTCGCCCGCCTGCAGTTCACC |
| | TACAACCACATCCAGCGCCACGTGAACGACATGCTGGGCAGGATCG |
| | CCGTGGCCTGGTGCGAGCTGCAGAACCACGAGCTGACCCTGTGGAA |
| | CGAGGCCCGGAAGCTGAACCCCAACGCCATCGCCTCCGCCACCGTG |
| | GGCAGGCGCGTGAGCGCCAGGATGCTGGGCGACGTGATGGCCGTGG |
| | CCACCTGCGTGCCCGTGGCACCCGACAACGTGATCGTGCAGAACAG |
| | CATGCGGGTGAGCAGCCGGCCCGGCACCTGCTACAGCAGGCCCCTG |
| | GTGTCCTTCCGCTACGAGGACCAGGGGCCACTGATCGAGGGCCAGC |
| | TGGGCGAGAACAACGACGTGCGGCTGACCAGGGACGCCCTGGAGCC |
| | CTGCACCGTGGGCCACAGGGGCTACTTCATCTTCGGCGGCGGCTAC |
| | GTGTACTTCGAGGAGTACGCCTACTCCCACCAGCTGTCCCGCGCCG |
| | ACGTGACCACCGTGAGCACCTTCATCGACCTGAACATCACCATGCT |
| | GGAGGACCACGAGTTCGTGCCCCTGGAGGTGTACACCCGGCACGAG |
| | ATCAAGGACTCCGGCCTGCTGGACTACACCGAGGTGCAGCGGAGGA |
| | ACCAGCTGCACGACCTGAGGTTCGCCGACATCGACACCGTGATCAG |
| | GGCCGACGCCAACGCCGCCATGTTCGCCGGCCTGTGCGCCTTCTTC |
| | GAGGGCATGGGCGACCTGGGCAGAGCCGTGGGCAAGGTGGTCATGG |
| | GCGTGGTGGGAGGCGTGGTGAGCGCCGTGTCCGGCGTGAGCAGCTT |
| | CATGTCCAACCCCTTCGGCGCCCTGGCCGTGGGACTGCTGGTGCTG |
| | GCCGGACTGGTGGCAGCCTTCTTCGCCTTCCGCTACGTGCTGCAGC |
| | TGCAGCGGAACCCCATGAAGGCCCTGTACCCCCTGACCACCAAGGA |
| | GCTGAAGACCAGCGACCCAGGCGGAGTCGGCGGAGAGGGCGAGGAG |
| | GGCGCCGAGGGCGGCGGCTTCGACGAGGCCAAGCTGGCCGAGGCCA |
| | GGGAGATGATCCGCTACATGGCCCTGGTGAGCGCCATGGAGCGGAC |
| | CGAGCACAAGGCCAGGAAGAAGGGCACAAGCGCCCTGCTGTCCAGC |
| | AAGGTGACCAACATGGTGCTGCGGAAGAGGAACAAGGCCAGGTACT |
| | CCCCACTGCACAACGAGGATGAGGCAGGCGACGAGGACGAGCTGTG |
| | A |
| HSV-2 gB-pf | MRGGGLICALVVGALVAAVASAAPAAPAAPRASGGVAATVAANGGP |
| mutant | ASRPPPVPSPATTKARKRKTKKPPKRPEATPPPDANATVAAGHATV |
| (aa sequence) | RAHLREIKVENADAQFYVCPPPTGATVVQFEQPRRCPTRPEGQNYT |
| (SEQ ID NO: 3) | EGIAVVFKENIAPYKFKATMYYKDVTVSQVWFGHRYSQFMGIFEDR |
| | APVPFEEVIDKINAKGVCRSTAKYVRNNMETTAFHRDDHETDMELK |
| | PAKVATRTSRGWHTTDLKYNPSRVEAFHRYGTTVNCIVEEVDARSV |
| | YPYDEFVLATGDFVYMSPFYGYREGSHTEHTTYAADRFKQVDGFYA |
| | RDLTTKARATSPTTRNLLTTPKFTVAWDWVPKRPAVCTMTKWQEVD |
| | EMLRAEYGGSFRFSSDAISTTFTTNLTEYSLSRVDLGDCIGRDARE |
| | AIDRMFARKYNATHIKVGQPQYYQATGGFLIAYQPLLSNTLAELYV |
| | REYMREQDRKPRNATPAPLREAPSANASVERIKTTSSIEFARLQFT |
| | YNHIQRPVNDMLGRIAVAWCELQNHELTLWNEARKLNPNAIASATV |
| | GRRVSARMLGDVMAVATCVPVAPDNVIVQNSMRVSSRPGTCYSRPL |
| | VSFRYEDQGPLIEGQLGENNDVRLTRDALEPCTVGHRGYFIFGGGY |
| | VYFEEYAYSHQLSRADVTTVSTFIDLNITMLEDHEFVPLEVYTRHE |
| | IKDSGLLDYTEVQRRNQLHDLRFADIDTVIRADANAAMFAGLCAFF |
| | EGMGDLGRAVGKVVMGVVGGVVSAVSGVSSFMSNPFGALAVGLLVL |
| | AGLVAAFFAFRYVLQLQRNPMKALYPLTTKELKTSDPGGVGGEGEE |
| | GAEGGGFDEAKLAEAREMIRYMALVSAMERTEHKARKKGTSALLSS |
| | KVTNMVLRKRNKARYSPLHNEDEAGDEDEL |
| HSV-2 gB-pf | ATGAGGGGCGGCGGCCTGATCTGCGCCCTGGTGGTGGGCGCCCTGG |
| mutant | TGGCCGCCGTGGCCAGCGCCGCACCAGCAGCACCAGCAGCCCCCAG |
| (DNA | GGCCTCCGGCGGCGTGGCCGCCACCGTGGCCGCCAACGGCGGCCCA |
| sequence) | GCCTCCCGCCCCCCACCAGTGCCAAGCCCCGCCACCACAAAGGCCA |
| (SEQ ID NO: 4) | GGAAGCGCAAGACAAAGAAGCCACCCAAGAGGCCAGAGGCAACCCC |
| | ACCCCCAGACGCAAACGCAACCGTGGCAGCAGGACACGCCACAGTG |
| | AGGGCCCACCTGCGCGAGATCAAGGTGGAGAACGCCGACGCCCAGT |
| | TCTACGTGTGCCCACCACCCACCGGAGCAACAGTGGTGCAGTTCGA |
| | GCAGCCACGGAGATGCCCAACCAGGCCAGAGGGCCAGAACTACACA |
| | GAGGGCATCGCCGTGGTGTTCAAGGAGAACATCGCCCCATACAAGT |
| | TCAAGGCCACCATGTACTACAAGGACGTGACAGTGAGCCAAGTGTG |
| | GTTCGGCCACCGCTACAGCCAGTTCATGGGCATCTTCGAGGACCGG |
| | GCCCCTGTGCCCTTCGAGGAAGTGATCGACAAGATCAACGCCAAGG |
| | GCGTGTGCAGGAGCACCGCCAAGTACGTGCGCAACAACATGGAGAC |
| | CACCGCCTTCCACAGGGACGACCACGAGACCGACATGGAGCTGAAG |
| | CCAGCCAAGGTGGCCACCAGGACCTCCCGCGGCTGGCACACCACCG |
| | ACCTGAAGTACAACCCCAGCCGGGTGGAGGCCTTCCACAGATATGG |
| | CACCACCGTGAACTGCATCGTGGAGGAGGTGGATGCCCGCTCCGTG |
| | TACCCCTACGACGAGTTCGTGCTGGCCACCGGCGACTTCGTGTACA |
| | TGAGCCCCTTCTACGGCTACCGGGAGGGCAGCCACACCGAGCACAC |
| | CACCTACGCCGCCGACAGGTTCAAGCAGGTGGACGGCTTCTACGCC |
| | CGGGACCTGACCACCAAGGCCAGGGCCACCAGCCCCACCACCAGGA |
| | ACCTGCTGACCACCCCAAAGTTCACCGTGGCCTGGGACTGGGTGCC |
| | CAAGAGGCCAGCCGTGTGCACCATGACCAAGTGGCAGGAGGTGGAC |
| | GAGATGCTGAGGGCCGAGTACGGCGGCTCCTTCAGGTTCAGCAGCG |
| | ACGCCATCAGCACCACCTTCACCACCAACCTGACCGAGTACAGCCT |
| | GAGCCGGGTGGACCTGGGCGACTGCATCGGAAGGGACGCCAGAGAG |
| | GCAATCGACCGGATGTTCGCCAGAAAGTACAACGCCACACACATCA |
| | AGGTCGGCCAGCCCCAGTACTACCAGGCCACCGGCGGCTTCCTGAT |
| | CGCCTACCAGCCCCTGCTGAGCAACACCCTGGCCGAGCTGTACGTG |
| | AGGGAGTACATGCGCGAGCAGGACAGGAAGCCCAGGAACGCCACCC |
| | CAGCACCCCTGAGGGAGGCACCCTCCGCCAACGCCAGCGTGGAGAG |
| | GATCAAGACCACCTCCAGCATCGAGTTCGCCCGCCTGCAGTTCACC |
| | TACAACCACATCCAGCGCCCAGTGAACGACATGCTGGGCAGGATCG |
| | CCGTGGCCTGGTGCGAGCTGCAGAACCACGAGCTGACCCTGTGGAA |
| | CGAGGCCCGGAAGCTGAACCCCAACGCCATCGCCTCCGCCACCGTG |
| | GGCAGGCGCGTGAGCGCCAGGATGCTGGGCGACGTGATGGCCGTGG |
| | CCACCTGCGTGCCCGTGGCACCCGACAACGTGATCGTGCAGAACAG |
| | CATGCGGGTGAGCAGCCGGCCCGGCACCTGCTACAGCAGGCCCCTG |
| | GTGTCCTTCCGCTACGAGGACCAGGGGCCACTGATCGAGGGCCAGC |
| | TGGGCGAGAACAACGACGTGCGGCTGACCAGGGACGCCCTGGAGCC |
| | CTGCACCGTGGGCCACAGGGGCTACTTCATCTTCGGCGGCGGCTAC |
| | GTGTACTTCGAGGAGTACGCCTACTCCCACCAGCTGTCCCGCGCCG |
| | ACGTGACCACCGTGAGCACCTTCATCGACCTGAACATCACCATGCT |
| | GGAGGACCACGAGTTCGTGCCCCTGGAGGTGTACACCCGGCACGAG |
| | ATCAAGGACTCCGGCCTGCTGGACTACACCGAGGTGCAGCGGAGGA |
| | ACCAGCTGCACGACCTGAGGTTCGCCGACATCGACACCGTGATCAG |
| | GGCCGACGCCAACGCCGCCATGTTCGCCGGCCTGTGCGCCTTCTTC |
| | GAGGGCATGGGCGACCTGGGCAGAGCCGTGGGCAAGGTGGTCATGG |
| | GCGTGGTGGGAGGCGTGGTGAGCGCCGTGTCCGGCGTGAGCAGCTT |
| | CATGTCCAACCCCTTCGGCGCCCTGGCCGTGGGACTGCTGGTGCTG |
| | GCCGGACTGGTGGCAGCCTTCTTCGCCTTCCGCTACGTGCTGCAGC |
| | TGCAGCGGAACCCCATGAAGGCCCTGTACCCCCTGACCACCAAGGA |
| | GCTGAAGACCAGCGACCCAGGCGGAGTCGGCGGAGAGGGCGAGGAG |
| | GGCGCCGAGGGCGGCGGCTTCGACGAGGCCAAGCTGGCCGAGGCCA |
| | GGGAGATGATCCGCTACATGGCCCTGGTGAGCGCCATGGAGCGGAC |
| | CGAGCACAAGGCCAGGAAGAAGGGCACAAGCGCCCTGCTGTCCAGC |
| | AAGGTGACCAACATGGTGCTGCGGAAGAGGAACAAGGCCAGGTACT |
| | CCCCACTGCACAACGAGGATGAGGCAGGCGACGAGGACGAGCTGTG |
| | A |
| HSV-1 gB-pf | MRQGAPARGRRWFVVWALLGLTLGVLVASAAPSSPGTPGVAAATQA |
| (aa sequence) | ANGGPATPAPPAPGAPPTGDPKPKKNRKPKPPKPPRPAGDNATVAA |
| (SEQ ID NO: 5) | GHATLREHLRDIKAENTDANFYVCPPPTGATVVQFEQPRRCPTRPE |
| | GQNYTEGIAVVFKENIAPYKFKATMYYKDVTVSQVWFGHRYSQFMG |
| | IFEDRAPVPFEEVIDKINAKGVCRSTAKYVRNNLETTAFHRDDHET |
| | DMELKPANAATRTSRGWHTTDLKYNPSRVEAFHRYGTTVNCIVEEV |
| | DARSVYPYDEFVLATGDFVYMSPFYGYREGSHTEHTSYAADRFKQV |
| | DGFYARDLTTKARATAPTTRNLLTTPKFTVAWDWVPKRPSVCTMTK |
| | WQEVDEMLRSEYGGSFRFSSDAISTTFTTNLTEYPLSRVDLGDCIG |
| | KDARDAMDRIFARRYNATHIKVGQPQYYLANGGFLIAYQPLLSNTL |
| | AELYVREHLREQSRKPPNPTPPPPGASANASVERIKTTSSIEFARL |
| | QFTYNHIQRHVNDMLGRVAIAWCELQNHELTLWNEARKLNPNAIAS |
| | ATVGRRVSARMLGDVMAVSTCVPVAADNVIVQNSMRISSRPGACYS |
| | RPLVSFRYEDQGPLVEGQLGENNELRLTRDAIEPCTVGHRRYFTFG |
| | GGYVYFEEYAYSHQLSRADITTVSTFIDLNITMLEDHEFVPLEVYT |
| | RHEIKDSGLLDYTEVQRRNQLHDLRFADIDTVIHADANAAMFAGLG |
| | AFFEGMGDLGRAVGKVVMGIVGGVVSAVSGVSSFMSNPFGALAVGL |
| | LVLAGLAAAFFAFRYVMRLQSNPMKALYPLTTKELKNPTNPDASGE |
| | GEEGGDFDEAKLAEAREMIRYMALVSAMERTEHKAKKKGTSALLSA |
| | KVTDMVMRKRRNTNYTQVPNKDGDADEDDL |
| HSV-1 gB-pf | MRQGAPARGRRWFVVWALLGLTLGVLVASAAPSSPGTPGVAAATQA |
| mutant | ANGGPATPAPPAPGAPPTGDPKPKKNRKPKPPKPPRPAGDNATVAA |
| (aa sequence) | GHATLREHLRDIKAENTDANFYVCPPPTGATVVQFEQPRRCPTRPE |
| (SEQ ID NO: 6) | GQNYTEGIAVVFKENIAPYKFKATMYYKDVTVSQVWFGHRYSQFMG |
| | IFEDRAPVPFEEVIDKINAKGVCRSTAKYVRNNLETTAFHRDDHET |
| | DMELKPANAATRTSRGWHTTDLKYNPSRVEAFHRYGTTVNCIVEEV |
| | DARSVYPYDEFVLATGDFVYMSPFYGYREGSHTEHTSYAADRFKQV |
| | DGFYARDLTTKARATAPTTRNLLTTPKFTVAWDWVPKRPSVCTMTK |
| | WQEVDEMLRSEYGGSFRFSSDAISTTFTTNLTEYPLSRVDLGDCIG |
| | KDARDAMDRIFARRYNATHIKVGQPQYYLANGGFLIAYQPLLSNTL |
| | AELYVREHLREQSRKPPNPTPPPPGASANASVERIKTTSSIEFARL |
| | QFTYNHIQRPVNDMLGRVAIAWCELQNHELTLWNEARKLNPNAIAS |
| | ATVGRRVSARMLGDVMAVSTCVPVAADNVIVQNSMRISSRPGACYS |
| | RPLVSFRYEDQGPLVEGQLGENNELRLTRDAIEPCTVGHRRYFTFG |
| | GGYVYFEEYAYSHQLSRADITTVSTFIDLNITMLEDHEFVPLEVYT |
| | RHEIKDSGLLDYTEVQRRNQLHDLRFADIDTVIHADANAAMFAGLG |
| | AFFEGMGDLGRAVGKVVMGIVGGVVSAVSGVSSFMSNPFGALAVGL |
| | LVLAGLAAAFFAFRYVMRLQSNPMKALYPLTTKELKNPTNPDASGE |
| | GEEGGDFDEAKLAEAREMIRYMALVSAMERTEHKAKKKGTSALLSA |
| | KVTDMVMRKRRNTNYTQVPNKDGDADEDDL |
| HSV-2 gD | MGRLTSGVGTAALLVVAVGLRVVCAKYALADPSLKMADPNRFRGKN |
| (aa sequence) | LPVLDQLTDPPGVKRVYHIQPSLEDPFQPPSIPITVYYAVLERACR |
| (SEQ ID NO: 7) | SVLLHAPSEAPQIVRGASDEARKHTYNLTIAWYRMGDNCAIPITVM |
| | EYTECPYNKSLGVCPIRTQPRWSYYDSFSAVSEDNLGFLMHAPAFE |
| | TAGTYLRLVKINDWTEITQFILEHRARASCKYALPLRIPPAACLTS |
| | KAYQQGVTVDSIGMLPRFIPENQRTVALYSLKIAGWHGPKPPYTST |
| | LLPPELSDTTNATQPELVPEDPEDSALLEDPAGTVSSQIPPNWHIP |
| | SIQDVAPHHAPAAPSNPGLIIGALAGSTLAVLVIGGIAFWVRRRAQ |
| | MAPKRLRLPHIRDDDAPPSHQPLFY |
| HSV-2 gD | ATGGGCAGACTGACCAGCGGAGTGGGCACAGCCGCCCTGCTGGTGG |
| (DNA | TGGCAGTGGGACTGAGGGTGGTGTGCGCAAAGTACGCCCTGGCCGA |
| sequence) | CCCATCCCTGAAGATGGCCGACCCCAACCGCTTCCGGGGCAAGAAC |
| (SEQ ID NO: 8) | CTGCCCGTGCTGGACCAGCTGACCGACCCCCCTGGCGTGAAGAGAG |
| | TGTACCACATCCAGCCCAGCCTGGAGGACCCCTTCCAGCCACCCAG |
| | CATCCCAATCACAGTGTACTACGCCGTGCTGGAGAGAGCCTGCAGG |
| | TCCGTGCTGCTGCACGCACCCAGCGAGGCACCACAGATCGTGAGAG |
| | GCGCCAGCGACGAGGCCAGGAAGCACACCTACAACCTGACCATCGC |
| | CTGGTACAGGATGGGCGACAACTGCGCCATCCCCATCACCGTGATG |
| | GAGTACACAGAGTGCCCATACAACAAGAGCCTGGGCGTGTGCCCCA |
| | TCAGGACCCAGCCACGGTGGAGCTACTACGACAGCTTCAGCGCCGT |
| | GAGCGAGGACAACCTGGGCTTCCTGATGCACGCACCAGCCTTCGAG |
| | ACCGCAGGCACATACCTGCGCCTGGTGAAGATCAATGATTGGACCG |
| | AGATCACACAGTTTATCCTGGAGCACAGAGCCAGGGCCTCCTGCAA |
| | GTACGCACTGCCCCTGAGAATCCCACCCGCAGCCTGCCTGACCAGC |
| | AAGGCCTACCAGCAGGGCGTGACAGTGGACTCCATCGGCATGCTGC |
| | CCCGCTTCATCCCAGAGAACCAGCGGACCGTGGCCCTGTACAGCCT |
| | GAAGATCGCAGGATGGCACGGACCAAAGCCACCCTACACCAGCACA |
| | CTGCTGCCACCCGAGCTGTCCGACACCACAAACGCCACCCAGCCAG |
| | AGCTGGTGCCAGAGGACCCCGAGGACAGCGCCCTGCTGGAGGACCC |
| | CGCAGGCACCGTGAGCAGCCAGATCCCACCCAACTGGCACATCCCC |
| | AGCATCCAGGACGTGGCACCACACCACGCACCCGCCGCACCAAGCA |
| | ACCCAGGCCTGATCATCGGCGCCCTGGCCGGCAGCACCCTGGCCGT |
| | GCTGGTCATCGGAGGAATCGCCTTCTGGGTGCGGAGAAGGGCCCAG |
| | ATGGCACCCAAGCGCCTGAGGCTGCCCCACATCAGGGACGACGACG |
| | CACCACCCAGCCACCAGCCCCTGTTCTACTGA |
| HSV-2 gD | KYALADPSLKMADPNRFRGKNLPVLDQLTDPPGVKRVYHIQPSLED |
| fragment | PFQPPSIPITVYYAVLERACRSVLLHAPSEAPQIVRGASDEARKHT |
| (aa sequence) | YNLTIAWYRMGDNCAIPITVMEYTECPYNKSLGVCPIRTQPRWSYY |
| (SEQ ID NO: 9) | DSFSAVSEDNLGFLMHAPAFETAGTYLRLVKINDWTEITQFILEHR |
| | ARASCKYALPLRIPPAACLTSKAYQQGVTVDSIGMLPRFIPENQRT |
| | VALYSLKIAGWHGPKPPYTSTLLPPELSDTTNATQPELVPEDPEDS |
| | ALLEDPAGTVSSQIPPNWHIPSIQDVAPHH |
| HSV-2 gE | MARGAGLVFFVGVWVVSCLAAAPRTSWKRVTSGEDVVLLPAPAERT |
| (aa sequence) | RAHKLLWAAEPLDACGPLRPSWVALWPPRRVLETVVDAACMRAPEP |
| (SEQ ID NO: 10) | LAIAYSPPFPAGDEGLYSELAWRDRVAVVNESLVIYGALETDSGLY |
| | TLSVVGLSDEARQVASVVLVVEPAPVPTPTPDDYDEEDDAGVTNAR |
| | RSAFPPQPPPRRPPVAPPTHPRVIPEVSHVRGVTVHMETLEAILFA |
| | PGETFGTNVSIHAIAHDDGPYAMDVVWMRFDVPSSCADMRIYEACL |
| | YHPQLPECLSPADAPCAVSSWAYRLAVRSYAGCSRTTPPPRCFAEA |
| | RMEPVPGLAWLASTVNLEFQHASPQHAGLYLCVVYVDDHIHAWGHM |
| | TISTAAQYRNAVVEQHLPQRQPEPVEPTRPHVRAPHPAPSARGPLR |
| | LGAVLGAALLLAALGLSAWACMTCWRRRSWRAVKSRASATGPTYIR |
| | VADSELYADWSSDSEGERDGSLWQDPPERPDSPSTNGSGFEILSPT |
| | APSVYPHSEGRKSRRPLTTFGSGSPGRRHSQASYPSVLW |
| HSV-2 gE | ATGGCCAGGGGCGCAGGACTGGTGTTCTTCGTGGGCGTGTGGGTGG |
| (DNA | TGTCCTGCCTGGCCGCCGCCCCACGCACCAGCTGGAAGAGGGTGAC |
| sequence) | AAGCGGAGAGGACGTGGTGCTGCTGCCAGCACCCGCAGAGAGAACC |
| (SEQ ID NO: 11) | AGGGCCCACAAGCTGCTGTGGGCAGCCGAGCCCCTGGACGCATGCG |
| | GACCCCTGAGGCCAAGCTGGGTGGCCCTGTGGCCACCCCGGAGAGT |
| | GCTGGAGACAGTGGTGGACGCAGCCTGCATGCGCGCCCCAGAGCCC |
| | CTGGCCATCGCATACAGCCCACCCTTCCCAGCAGGCGACGAGGGAC |
| | TGTACAGCGAGCTGGCCTGGAGGGACAGGGTGGCAGTGGTGAACGA |
| | GAGCCTGGTCATCTACGGCGCCCTGGAGACCGACAGCGGACTGTAC |
| | ACACTGAGCGTGGTGGGCCTGTCCGACGAGGCCAGACAGGTGGCCA |
| | GCGTGGTGCTGGTGGTGGAGCCCGCACCAGTGCCCACCCCCACACC |
| | AGACGACTACGACGAGGAGGACGACGCAGGAGTGACCAACGCAAGG |
| | CGCAGCGCCTTCCCACCCCAGCCCCCACCACGGAGACCCCCAGTCG |
| | CACCACCAACACACCCCAGAGTGATCCCAGAGGTGAGCCACGTGCG |
| | CGGAGTGACCGTGCACATGGAGACACTGGAGGCAATCCTGTTCGCA |
| | CCAGGAGAGACCTTCGGCACCAACGTGAGCATCCACGCAATCGCAC |
| | ACGACGACGGACCCTACGCCATGGACGTGGTGTGGATGCGGTTCGA |
| | CGTGCCCAGCAGCTGCGCCGACATGAGAATCTACGAGGCCTGCCTG |
| | TACCACCCACAGCTGCCAGAGTGCCTGAGCCCTGCAGACGCACCAT |
| | GCGCCGTGAGCAGCTGGGCCTACAGACTGGCCGTGAGGAGCTACGC |
| | CGGATGCAGCCGCACCACACCTCCACCACGGTGCTTCGCAGAGGCA |
| | AGAATGGAGCCCGTGCCCGGCCTGGCCTGGCTGGCCAGCACCGTGA |
| | ACCTGGAGTTCCAGCACGCCTCCCCACAGCACGCAGGACTGTACCT |
| | GTGCGTGGTGTACGTGGACGACCACATCCACGCCTGGGGCCACATG |
| | ACCATCAGCACAGCCGCCCAGTACAGGAACGCAGTGGTGGAGCAGC |
| | ACCTGCCACAGAGGCAGCCAGAGCCAGTGGAGCCAACCCGCCCCCA |
| | CGTGCGGGCACCACACCCAGCCCCCAGCGCCAGAGGCCCACTGAGG |
| | CTGGGAGCCGTGCTGGGAGCAGCCCTGCTGCTGGCCGCCCTGGGAC |
| | TGAGCGCCTGGGCATGCATGACCTGCTGGAGGCGCCGGAGCTGGAG |
| | AGCAGTGAAGTCCAGGGCCAGCGCCACCGGACCAACATACATCAGG |
| | GTGGCAGACAGCGAGCTGTACGCCGACTGGAGCAGCGACAGCGAGG |
| | GAGAGAGGGACGGCAGCCTGTGGCAGGACCCACCCGAGAGACCCGA |
| | CAGCCCATCCACCAACGGCAGCGGCTTCGAGATCCTGAGCCCCACA |
| | GCACCCAGCGTGTACCCACACAGCGAGGGAAGAAAGTCCAGAAGGC |
| | CCCTGACCACATTCGGCAGCGGCAGCCCAGGACGCCGGCACAGCCA |
| | GGCCAGCTACCCAAGCGTGCTGTGGTGA |
| HSV-2 gE | RTSWKRVTSGEDVVLLPAPAGPEERTRAHKLLWAAEPLDACGPLRP |
| fragment | SWVALWPPRRVLETVVDAACMRAPEPLAIAYSPPFPAGDEGLYSEL |
| (aa sequence) | AWRDRVAVVNESLVIYGALETDSGLYTLSVVGLSDEARQVASVVLV |
| (SEQ ID NO: 12) | VEPAPVPTPTPDDYDEEDDAGVSERTPVSVPPPTPPRRPPVAPPTH |
| | PRVIPEVSHVRGVTVHMETPEAILFAPGETFGTNVSIHAIAHDDGP |
| | YAMDVVWMRFDVPSSCAEMRIYEACLYHPQLPECLSPADAPCAVSS |
| | WAYRLAVRSYAGCSRTTPPPRCFAEARMEPVPGLAWLASTVNLEFQ |
| | HASPQHAGLYLCVVYVDDHIHAWGHMTISTAAQYRNAVVEQHLPQR |
| | QPEPVEPTRPHVRA |
| HSV-2 gC | MGAGVPWTGIKARGAGGPITVRVLGWEVAQKATLPCCSCPREAVVS |
| (aa sequence) | GNPPRCAGRAHRSFAGAGALLVMALGRVGLAVGLWGLLWVGVVVVL |
| (SEQ ID NO: 13) | ANASPGRTITVGPRGNASNAAPSASPRNASAPRTTPTPPQPRKATK |
| | SKASTAKPAPPPKTGPPKTSSEPVRCNRHDPLARYGSRVQIRCRFP |
| | NSTRTEFRLQIWRYATATDAEIGTAPSLEEVMVNVSAPPGGQLVYD |
| | SAPNRTDPHVIWAEGAGPGASPRLYSVVGPLGRQRLIIEELTLETQ |
| | GMYYWVWGRTDRPSAYGTWVRVRVFRPPSLTIHPHAVLEGQPFKAT |
| | CTAATYYPGNRAEFVWFEDGRRVFDPAQIHTQTQENPDGFSTVSTV |
| | TSAAVGGQGPPRTFTCQLTWHRDSVSFSRRNASGTASVLPRPTITM |
| | EFTGDHAVCTAGCVPEGVTFAWFLGDDSSPAEKVAVASQTSCGRPG |
| | TATIRSTLPVSYEQTEYICRLAGYPDGIPVLEHHGSHQPPPRDPTE |
| | RQVIRAVEGAGIGVAVLVAVVLAGTAVVYLTHASSVRYRRLR |
| HSV-2 gC | ATGGCCCTGGGAAGGGTGGGCCTGGCCGTGGGCCTGTGGGGCCTGC |
| (DNA | TGTGGGTGGGAGTGGTGGTGGTGCTGGCCAACGCCTCCCCAGGAAG |
| sequence) | GACCATCACCGTGGGACCACGCGGCAACGCCAGCAACGCCGCACCA |
| (SEQ ID NO: 14) | AGCGCCTCCCCAAGGAACGCCTCCGCCCCAAGGACCACACCCACCC |
| | CACCACAGCCAAGGAAGGCCACCAAGAGCAAGGCCAGCACAGCAAA |
| | GCCAGCACCACCACCCAAGACCGGACCACCCAAGACCAGCTCCGAG |
| | CCCGTGCGGTGCAACAGACACGACCCACTGGCCCGGTACGGCTCCA |
| | GAGTGCAGATCAGGTGCCGCTTCCCCAACAGCACCAGGACAGAGTT |
| | CCGCCTGCAAATCTGGCGGTACGCCACCGCAACCGACGCAGAGATC |
| | GGCACCGCACCAAGCCTGGAGGAAGTGATGGTGAACGTGAGCGCCC |
| | CACCAGGAGGACAGCTGGTGTACGACAGCGCCCCAAACCGGACCGA |
| | CCCACACGTGATCTGGGCAGAGGGAGCAGGACCCGGAGCCTCCCCA |
| | AGACTGTACAGCGTGGTGGGACCACTGGGCCGGCAGAGACTGATCA |
| | TCGAGGAGCTGACCCTGGAGACACAGGGCATGTACTACTGGGTGTG |
| | GGGAAGGACCGACAGGCCAAGCGCCTACGGCACATGGGTGAGGGTG |
| | CGCGTGTTCCGGCCACCCAGCCTGACCATCCACCCACACGCCGTGC |
| | TGGAGGGACAGCCCTTCAAGGCCACCTGCACAGCCGCCACATACTA |
| | CCCCGGCAACAGAGCCGAGTTCGTGTGGTTCGAGGACGGCCGGAGA |
| | GTGTTCGACCCAGCCCAGATCCACACCCAGACACAGGAGAACCCAG |
| | ACGGCTTCTCCACCGTGAGCACCGTGACATCCGCCGCAGTGGGAGG |
| | ACAGGGACCACCAAGGACCTTCACATGCCAGCTGACCTGGCACCGC |
| | GACTCTGTGAGCTTTTCCAGGCGCAACGCCTCTGGCACAGCCAGCG |
| | TGCTGCCTAGGCCAACCATCACCATGGAGTTCACCGGCGACCACGC |
| | CGTGTGCACAGCAGGATGCGTGCCAGAGGGAGTGACCTTCGCCTGG |
| | TTCCTGGGCGACGACAGCAGCCCAGCAGAGAAGGTGGCAGTGGCCA |
| | GCCAGACATCCTGCGGCCGGCCAGGCACCGCAACAATCAGAAGCAC |
| | CCTGCCAGTGAGCTACGAGCAGACAGAGTACATCTGCAGGCTGGCC |
| | GGCTACCCCGACGGCATCCCAGTGCTGGAGCACCACGGCAGCCACC |
| | AGCCACCACCCCGCGACCCCACCGAGAGGCAGGTCATCAGAGCAGT |
| | GGAGGGAGCAGGAATCGGAGTGGCCGTGCTGGTGGCCGTGGTGCTG |
| | GCCGGCACCGCAGTGGTGTACCTGACACACGCCAGCAGCGTGAGGT |
| | ACAGGAGACTGCGCTGA |
| HSV-2 gC2 | ASPGRTITVGPRGNASNAAPSASPRNASAPRTTPTPPQPRKATKSK |
| fragment | ASTAKPAPPPKTGPPKTSSEPVRCNRHDPLARYGSRVQIRCRFPNS |
| (aa sequence) | TRTESRLQIWRYATATDAEIGTAPSLEEVMVNVSAPPGGQLVYDSA |
| (SEQ ID NO: 15) | PNRTDPHVIWAEGAGPGASPRLYSVVGPLGRQRLIIEELTLETQGM |
| | YYWVWGRTDRPSAYGTWVRVRVFRPPSLTIHPHAVLEGQPFKATCT |
| | AATYYPGNRAEFVWFEDGRRVFDPAQIHTQTQENPDGFSTVSTVTS |
| | AAVGGQGPPRTFTCQLTWHRDSVSFSRRNASGTASVLPRPTITMEF |
| | TGDHAVCTAGCVPEGVTFAWFLGDDSSPAEKVAVASQTSCGRPGTA |
| | TIRSTLPVSYEQTEYICRLAGYPDGIPVLEHH |

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

1. An immunogenic composition comprising:
(a) mRNA encoding herpesvirus glycoprotein D or an immunogenic fragment thereof and mRNA encoding herpesvirus glycoprotein B or an immunogenic fragment thereof, wherein the mRNA for herpesvirus glycoprotein B is translated to glycoprotein B in prefusion fixed form; and
(b) optionally, an adjuvant.

2. The composition of claim 1, wherein the composition comprises mRNA encoding glycoprotein D.

3. The composition of claim 1, wherein the composition comprises mRNA encoding the extracellular domain region for herpesvirus glycoprotein D.

4. The composition of claim 1, wherein the composition comprises mRNA encoding glycoprotein B.

5. The composition of claim 1, wherein the composition comprises mRNA encoding the extracellular domain region for herpesvirus glycoprotein B in prefusion fixed form.

6. The composition of claim 1, wherein the composition comprises mRNA encoding a mutated herpesvirus glycoprotein B or its extracellular domain region, which is stable in its pre-fusion form and does not convert to a post-fusion form upon interaction with a cellular membrane.

7. The composition of claim 1, wherein the composition further comprises (i) mRNA encoding herpesvirus glycoprotein C or an immunogenic fragment thereof, (ii) mRNA encoding herpesvirus glycoprotein E or an immunogenic fragment thereof, or (iii) both.

8. The composition of claim 1, wherein the composition is suitable for systemic, intramuscular, intradermal, subcutaneous, intravaginal, or parenteral administration or administration by intracerebroventricular or intraperitoneal injection.

9. The composition of claim 1, wherein the mRNA are complexed with polymeric or lipid components, encapsulated in liposomes, or in lipid nanoparticles.

10. The composition of claim 1, wherein the herpesvirus is one of herpes simplex virus 1 or herpes simplex virus 2.

11. The composition of claim 1, wherein the composition is administered to a subject at least twice.

12. A method of inducing an anti-HSV immune response in a mammalian subject comprising administering to the subject an effective amount of the immunogenic composition of claim 1.

13. The method of claim 12, further comprising the step of administering to the subject a booster vaccination which comprises the immunogenic composition of claim 1.

14. A method of suppressing, inhibiting, or reducing an incidence of an HSV infection in a subject comprising administering to the subject an effective amount of the immunogenic composition of claim 1.

15. The method of claim 14, wherein the HSV infection is an HSV-1 infection.

16. The method of claim 14, wherein the HSV infection is an HSV-2 infection.


SEQUENCE LISTING (17 sequences)

SEQ ID NO: 1 - protein, 904 aa, Human alphaherpesvirus 2
MRGGGLICALVVGALVAAVASAAPAAPAAPRASGGVAATVAANGGPASRPPPVPSPATTKARKRKTKKPPKRPEATPPPDANATVAAGHATVRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYQATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVATCVPVAPDNVIVQNSMRVSSRPGTCYSRPLVSFRYEDQGPLIEGQLGENNDVRLTRDALEPCTVGHRGYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 2 - DNA, 2715 bp, Human alphaherpesvirus 2
  source 1..2715: mol_type = genomic DNA
atgaggggcggcggcctgatctgcgccctggtggtgggcgccctggtggccgccgtggccagcgccgcaccagcagcaccagcagcccccagggcctccggcggcgtggccgccaccgtggccgccaacggcggcccagcctcccgccccccaccagtgccaagccccgccaccacaaaggccaggaagcgcaagacaaagaagccacccaagaggccagaggcaaccccacccccagacgcaaacgcaaccgtggcagcaggacacgccacagtgagggcccacctgcgcgagatcaaggtggagaacgccgacgcccagttctacgtgtgcccaccacccaccggagcaacagtggtgcagttcgagcagccacggagatgcccaaccaggccagagggccagaactacacagagggcatcgccgtggtgttcaaggagaacatcgccccatacaagttcaaggccaccatgtactacaaggacgtgacagtgagccaagtgtggttcggccaccgctacagccagttcatgggcatcttcgaggaccgggcccctgtgcccttcgaggaagtgatcgacaagatcaacgccaagggcgtgtgcaggagcaccgccaagtacgtgcgcaacaacatggagaccaccgccttccacagggacgaccacgagaccgacatggagctgaagccagccaaggtggccaccaggacctcccgcggctggcacaccaccgacctgaagtacaaccccagccgggtggaggccttccacagatatggcaccaccgtgaactgcatcgtggaggaggtggatgcccgctccgtgtacccctacgacgagttcgtgctggccaccggcgacttcgtgtacatgagccccttctacggctaccgggagggcagccacaccgagcacaccacctacgccgccgacaggttcaagcaggtggacggcttctacgcccgggacctgaccaccaaggccagggccaccagccccaccaccaggaacctgctgaccaccccaaagttcaccgtggcctgggactgggtgcccaagaggccagccgtgtgcaccatgaccaagtggcaggaggtggacgagatgctgagggccgagtacggcggctccttcaggttcagcagcgacgccatcagcaccaccttcaccaccaacctgaccgagtacagcctgagccgggtggacctgggcgactgcatcggaagggacgccagagaggcaatcgaccggatgttcgccagaaagtacaacgccacacacatcaaggtcggccagccccagtactaccaggccaccggcggcttcctgatcgcctaccagcccctgctgagcaacaccctggccgagctgtacgtgagggagtacatgcgcgagcaggacaggaagcccaggaacgccaccccagcacccctgagggaggcaccctccgccaacgccagcgtggagaggatcaagaccacctccagcatcgagttcgcccgcctgcagttcacctacaaccacatccagcgccacgtgaacgacatgctgggcaggatcgccgtggcctggtgcgagctgcagaaccacgagctgaccctgtggaacgaggcccggaagctgaaccccaacgccatcgcctccgccaccgtgggcaggcgcgtgagcgccaggatgctgggcgacgtgatggccgtggccacctgcgtgcccgtggcacccgacaacgtgatcgtgcagaacagcatgcgggtgagcagccggcccggcacctgctacagcaggcccctggtgtccttccgctacgaggaccaggggccactgatcgagggccagctgggcgagaacaacgacgtgcggctgaccagggacgccctggagccctgcaccgtgggccacaggggctacttcatcttcggcggcggctacgtgtacttcgaggagtacgcctactcccaccagctgtcccgcgccgacgtgaccaccgtgagcaccttcatcgacctgaacatcaccatgctggaggaccacgagttcgtgcccctggaggtgtacacccggcacgagatcaaggactccggcctgctggactacaccgaggtgcagcggaggaaccagctgcacgacctgaggttcgccgacatcgacaccgtgatcagggccgacgccaacgccgccatgttcgccggcctgtgcgccttcttcgagggcatgggcgacctgggcagagccgtgggcaaggtggtcatgggcgtggtgggaggcgtggtgagcgccgtgtccggcgtgagcagcttcatgtccaaccccttcggcgccctggccgtgggactgctggtgctggccggactggtggcagccttcttcgccttccgctacgtgctgcagctgcagcggaaccccatgaaggccctgtaccccctgaccaccaaggagctgaagaccagcgacccaggcggagtcggcggagagggcgaggagggcgccgagggcggcggcttcgacgaggccaagctggccgaggccagggagatgatccgctacatggccctggtgagcgccatggagcggaccgagcacaaggccaggaagaagggcacaagcgccctgctgtccagcaaggtgaccaacatggtgctgcggaagaggaacaaggccaggtactccccactgcacaacgaggatgaggcaggcgacgaggacgagctgtga

SEQ ID NO: 3 - protein, 904 aa, synthetic construct
MRGGGLICALVVGALVAAVASAAPAAPAAPRASGGVAATVAANGGPASRPPPVPSPATTKARKRKTKKPPKRPEATPPPDANATVAAGHATVRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYQATGGFLIAYQPLLSNTLAELYVREYMREQDRKPRNATPAPLREAPSANASVERIKTTSSIEFARLQFTYNHIQRPVNDMLGRIAVAWCELQNHELTLWNEARKLNPNAIASATVGRRVSARMLGDVMAVATCVPVAPDNVIVQNSMRVSSRPGTCYSRPLVSFRYEDQGPLIEGQLGENNDVRLTRDALEPCTVGHRGYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 4 - DNA, 2715 bp, synthetic construct
  source 1..2715: mol_type = other DNA
atgaggggcggcggcctgatctgcgccctggtggtgggcgccctggtggccgccgtggccagcgccgcaccagcagcaccagcagcccccagggcctccggcggcgtggccgccaccgtggccgccaacggcggcccagcctcccgccccccaccagtgccaagccccgccaccacaaaggccaggaagcgcaagacaaagaagccacccaagaggccagaggcaaccccacccccagacgcaaacgcaaccgtggcagcaggacacgccacagtgagggcccacctgcgcgagatcaaggtggagaacgccgacgcccagttctacgtgtgcccaccacccaccggagcaacagtggtgcagttcgagcagccacggagatgcccaaccaggccagagggccagaactacacagagggcatcgccgtggtgttcaaggagaacatcgccccatacaagttcaaggccaccatgtactacaaggacgtgacagtgagccaagtgtggttcggccaccgctacagccagttcatgggcatcttcgaggaccgggcccctgtgcccttcgaggaagtgatcgacaagatcaacgccaagggcgtgtgcaggagcaccgccaagtacgtgcgcaacaacatggagaccaccgccttccacagggacgaccacgagaccgacatggagctgaagccagccaaggtggccaccaggacctcccgcggctggcacaccaccgacctgaagtacaaccccagccgggtggaggccttccacagatatggcaccaccgtgaactgcatcgtggaggaggtggatgcccgctccgtgtacccctacgacgagttcgtgctggccaccggcgacttcgtgtacatgagccccttctacggctaccgggagggcagccacaccgagcacaccacctacgccgccgacaggttcaagcaggtggacggcttctacgcccgggacctgaccaccaaggccagggccaccagccccaccaccaggaacctgctgaccaccccaaagttcaccgtggcctgggactgggtgcccaagaggccagccgtgtgcaccatgaccaagtggcaggaggtggacgagatgctgagggccgagtacggcggctccttcaggttcagcagcgacgccatcagcaccaccttcaccaccaacctgaccgagtacagcctgagccgggtggacctgggcgactgcatcggaagggacgccagagaggcaatcgaccggatgttcgccagaaagtacaacgccacacacatcaaggtcggccagccccagtactaccaggccaccggcggcttcctgatcgcctaccagcccctgctgagcaacaccctggccgagctgtacgtgagggagtacatgcgcgagcaggacaggaagcccaggaacgccaccccagcacccctgagggaggcaccctccgccaacgccagcgtggagaggatcaagaccacctccagcatcgagttcgcccgcctgcagttcacctacaaccacatccagcgcccagtgaacgacatgctgggcaggatcgccgtggcctggtgcgagctgcagaaccacgagctgaccctgtggaacgaggcccggaagctgaaccccaacgccatcgcctccgccaccgtgggcaggcgcgtgagcgccaggatgctgggcgacgtgatggccgtggccacctgcgtgcccgtggcacccgacaacgtgatcgtgcagaacagcatgcgggtgagcagccggcccggcacctgctacagcaggcccctggtgtccttccgctacgaggaccaggggccactgatcgagggccagctgggcgagaacaacgacgtgcggctgaccagggacgccctggagccctgcaccgtgggccacaggggctacttcatcttcggcggcggctacgtgtacttcgaggagtacgcctactcccaccagctgtcccgcgccgacgtgaccaccgtgagcaccttcatcgacctgaacatcaccatgctggaggaccacgagttcgtgcccctggaggtgtacacccggcacgagatcaaggactccggcctgctggactacaccgaggtgcagcggaggaaccagctgcacgacctgaggttcgccgacatcgacaccgtgatcagggccgacgccaacgccgccatgttcgccggcctgtgcgccttcttcgagggcatgggcgacctgggcagagccgtgggcaaggtggtcatgggcgtggtgggaggcgtggtgagcgccgtgtccggcgtgagcagcttcatgtccaaccccttcggcgccctggccgtgggactgctggtgctggccggactggtggcagccttcttcgccttccgctacgtgctgcagctgcagcggaaccccatgaaggccctgtaccccctgaccaccaaggagctgaagaccagcgacccaggcggagtcggcggagagggcgaggagggcgccgagggcggcggcttcgacgaggccaagctggccgaggccagggagatgatccgctacatggccctggtgagcgccatggagcggaccgagcacaaggccaggaagaagggcacaagcgccctgctgtccagcaaggtgaccaacatggtgctgcggaagaggaacaaggccaggtactccccactgcacaacgaggatgaggcaggcgacgaggacgagctgtga

SEQ ID NO: 5 - protein, 904 aa, Human alphaherpesvirus 1
MRQGAPARGRRWFVVWALLGLTLGVLVASAAPSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 6 - protein, 904 aa, synthetic construct
MRQGAPARGRRWFVVWALLGLTLGVLVASAAPSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRPVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 7 - protein, 393 aa, Human alphaherpesvirus 2
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 8 - DNA, 1182 bp, Human alphaherpesvirus 2
  source 1..1182: mol_type = genomic DNA
atgggcagactgaccagcggagtgggcacagccgccctgctggtggtggcagtgggactgagggtggtgtgcgcaaagtacgccctggccgacccatccctgaagatggccgaccccaaccgcttccggggcaagaacctgcccgtgctggaccagctgaccgacccccctggcgtgaagagagtgtaccacatccagcccagcctggaggaccccttccagccacccagcatcccaatcacagtgtactacgccgtgctggagagagcctgcaggtccgtgctgctgcacgcacccagcgaggcaccacagatcgtgagaggcgccagcgacgaggccaggaagcacacctacaacctgaccatcgcctggtacaggatgggcgacaactgcgccatccccatcaccgtgatggagtacacagagtgcccatacaacaagagcctgggcgtgtgccccatcaggacccagccacggtggagctactacgacagcttcagcgccgtgagcgaggacaacctgggcttcctgatgcacgcaccagccttcgagaccgcaggcacatacctgcgcctggtgaagatcaatgattggaccgagatcacacagtttatcctggagcacagagccagggcctcctgcaagtacgcactgcccctgagaatcccacccgcagcctgcctgaccagcaaggcctaccagcagggcgtgacagtggactccatcggcatgctgccccgcttcatcccagagaaccagcggaccgtggccctgtacagcctgaagatcgcaggatggcacggaccaaagccaccctacaccagcacactgctgccacccgagctgtccgacaccacaaacgccacccagccagagctggtgccagaggaccccgaggacagcgccctgctggaggaccccgcaggcaccgtgagcagccagatcccacccaactggcacatccccagcatccaggacgtggcaccacaccacgcacccgccgcaccaagcaacccaggcctgatcatcggcgccctggccggcagcaccctggccgtgctggtcatcggaggaatcgccttctgggtgcggagaagggcccagatggcacccaagcgcctgaggctgccccacatcagggacgacgacgcaccacccagccaccagcccctgttctactga

SEQ ID NO: 9 - protein, 306 aa, synthetic construct
KYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 10 - protein, 545 aa, Human alphaherpesvirus 2
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVTNARRSAFPPQPPPRRPPVAPPTHPRVIPEVSHVRGVTVHMETLEAILFAPGETFGTNVSIHAIAHDDGPYAMDVVWMRFDVPSSCADMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYPSVLW

SEQ ID NO: 11 - DNA, 1638 bp, Human alphaherpesvirus 2
  source 1..1638: mol_type = genomic DNA
atggccaggggcgcaggactggtgttcttcgtgggcgtgtgggtggtgtcctgcctggccgccgccccacgcaccagctggaagagggtgacaagcggagaggacgtggtgctgctgccagcacccgcagagagaaccagggcccacaagctgctgtgggcagccgagcccctggacgcatgcggacccctgaggccaagctgggtggccctgtggccaccccggagagtgctggagacagtggtggacgcagcctgcatgcgcgccccagagcccctggccatcgcatacagcccacccttcccagcaggcgacgagggactgtacagcgagctggcctggagggacagggtggcagtggtgaacgagagcctggtcatctacggcgccctggagaccgacagcggactgtacacactgagcgtggtgggcctgtccgacgaggccagacaggtggccagcgtggtgctggtggtggagcccgcaccagtgcccacccccacaccagacgactacgacgaggaggacgacgcaggagtgaccaacgcaaggcgcagcgccttcccaccccagcccccaccacggagacccccagtcgcaccaccaacacaccccagagtgatcccagaggtgagccacgtgcgcggagtgaccgtgcacatggagacactggaggcaatcctgttcgcaccaggagagaccttcggcaccaacgtgagcatccacgcaatcgcacacgacgacggaccctacgccatggacgtggtgtggatgcggttcgacgtgcccagcagctgcgccgacatgagaatctacgaggcctgcctgtaccacccacagctgccagagtgcctgagccctgcagacgcaccatgcgccgtgagcagctgggcctacagactggccgtgaggagctacgccggatgcagccgcaccacacctccaccacggtgcttcgcagaggcaagaatggagcccgtgcccggcctggcctggctggccagcaccgtgaacctggagttccagcacgcctccccacagcacgcaggactgtacctgtgcgtggtgtacgtggacgaccacatccacgcctggggccacatgaccatcagcacagccgcccagtacaggaacgcagtggtggagcagcacctgccacagaggcagccagagccagtggagccaacccgcccccacgtgcgggcaccacacccagcccccagcgccagaggcccactgaggctgggagccgtgctgggagcagccctgctgctggccgccctgggactgagcgcctgggcatgcatgacctgctggaggcgccggagctggagagcagtgaagtccagggccagcgccaccggaccaacatacatcagggtggcagacagcgagctgtacgccgactggagcagcgacagcgagggagagagggacggcagcctgtggcaggacccacccgagagacccgacagcccatccaccaacggcagcggcttcgagatcctgagccccacagcacccagcgtgtacccacacagcgagggaagaaagtccagaaggcccctgaccacattcggcagcggcagcccaggacgccggcacagccaggccagctacccaagcgtgctgtggtga

SEQ ID NO: 12 - protein, 382 aa, synthetic construct
RTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 13 - protein, 548 aa, Human alphaherpesvirus 2
MGAGVPWTGIKARGAGGPITVRVLGWEVAQKATLPCCSCPREAVVSGNPPRCAGRAHRSFAGAGALLVMALGRVGLAVGLWGLLWVGVVVVLANASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHHGSHQPPPRDPTERQVIRAVEGAGIGVAVLVAVVLAGTAVVYLTHASSVRYRRLR

SEQ ID NO: 14 - DNA, 1443 bp, Human alphaherpesvirus 2
  source 1..1443: mol_type = genomic DNA
atggccctgggaagggtgggcctggccgtgggcctgtggggcctgctgtgggtgggagtggtggtggtgctggccaacgcctccccaggaaggaccatcaccgtgggaccacgcggcaacgccagcaacgccgcaccaagcgcctccccaaggaacgcctccgccccaaggaccacacccaccccaccacagccaaggaaggccaccaagagcaaggccagcacagcaaagccagcaccaccacccaagaccggaccacccaagaccagctccgagcccgtgcggtgcaacagacacgacccactggcccggtacggctccagagtgcagatcaggtgccgcttccccaacagcaccaggacagagttccgcctgcaaatctggcggtacgccaccgcaaccgacgcagagatcggcaccgcaccaagcctggaggaagtgatggtgaacgtgagcgccccaccaggaggacagctggtgtacgacagcgccccaaaccggaccgacccacacgtgatctgggcagagggagcaggacccggagcctccccaagactgtacagcgtggtgggaccactgggccggcagagactgatcatcgaggagctgaccctggagacacagggcatgtactactgggtgtggggaaggaccgacaggccaagcgcctacggcacatgggtgagggtgcgcgtgttccggccacccagcctgaccatccacccacacgccgtgctggagggacagcccttcaaggccacctgcacagccgccacatactaccccggcaacagagccgagttcgtgtggttcgaggacggccggagagtgttcgacccagcccagatccacacccagacacaggagaacccagacggcttctccaccgtgagcaccgtgacatccgccgcagtgggaggacagggaccaccaaggaccttcacatgccagctgacctggcaccgcgactctgtgagcttttccaggcgcaacgcctctggcacagccagcgtgctgcctaggccaaccatcaccatggagttcaccggcgaccacgccgtgtgcacagcaggatgcgtgccagagggagtgaccttcgcctggttcctgggcgacgacagcagcccagcagagaaggtggcagtggccagccagacatcctgcggccggccaggcaccgcaacaatcagaagcaccctgccagtgagctacgagcagacagagtacatctgcaggctggccggctaccccgacggcatcccagtgctggagcaccacggcagccaccagccaccaccccgcgaccccaccgagaggcaggtcatcagagcagtggagggagcaggaatcggagtggccgtgctggtggccgtggtgctggccggcaccgcagtggtgtacctgacacacgccagcagcgtgaggtacaggagactgcgctga

SEQ ID NO: 15 - protein, 400 aa, synthetic construct
ASPGRTITVGPRGNASNAAPSASPRNASAPRTTPTPPQPRKATKSKASTAKPAPPPKTGPPKTSSEPVRCNRHDPLARYGSRVQIRCRFPNSTRTESRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 16 - DNA, 24 bp, synthetic construct
  source 1..24: mol_type = other DNA
tcgtcgttttgtcgttttgtcgtt

SEQ ID NO: 17 - DNA, 20 bp, synthetic construct
  source 1..20: mol_type = other DNA
gggggacgatcgtcgggggg